(19) United States
(12) Patent Application Publication
(10) Pub. No.: US 2008/0200460 A1
(43) Pub. Date: Aug. 21, 2008

(54) Chemical Compounds

(76) Inventors: Dearg Brown, Cheshire (GB); John Oldfield, Cheshire (GB); Howard Tucker, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 11/793,436

(22) PCT Filed: Dec. 15, 2005
(86) PCT No.: PCT/GB05/04841

§ 371 Date: Jun. 18, 2007

(30) Foreign Application Priority Data
Dec. 20, 2004 (SE) 0403106-8

(51) Int. Cl. A61K 31/5377; C07D 401/14; A61K 31/496; C07D 221/00; A61P 37/00; A61K 31/4545; C07D 295/00

(52) U.S. Cl. 514/235.5; 544/360; 514/253.01; 546/189; 514/316; 546/190; 544/129

(57) ABSTRACT

Compounds of formula (I):

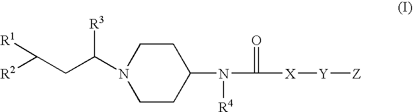

wherein:
when X is NR⁵, Y is absent or is CH₂;
when X is CH₂, Y is absent, CH₂, NR⁶, O, S, S(O) or S(O)₂;
Z is a 5- or 6-membered heterocyclyl ring;
   compositions comprising them, processes for preparing them and their use in medical therapy (for example modulating CCR5 receptor activity in a warm blooded animal).

The present invention relates to heterocyclic derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in PCT/SE01/01053, EP-A1-1013276, WO00/08013, WO99/38514 and WO99/04794.

Chemokines are chemotactic cytokines that are released by a wide variety of cells to attract macrophages, T cells, eosinophils, basophils and neutrophils to sites of inflammation and also play a rôle in the maturation of cells of the immune system. Chemokines play an important rôle in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C, or α) and Cys-Cys (C—C, or β) families. These are distinguished on the basis of a single amino acid insertion between the NH-proximal pair of cysteine residues and sequence similarity.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

The CCR5 receptor is expressed on T-lymphocytes, monocytes, macrophages, dendritic cells, microglia and other cell types. These detect and respond to several chemokines, principally “regulated on activation normal T-cell expressed and secreted” (RANTES), macrophage inflammatory proteins (MIP) MIP-1α and MIP-1β and monocyte chemoattractant protein-2 (MCP-2).

This results in the recruitment of cells of the immune system to sites of disease. In many diseases it is the cells expressing CCR5 which contribute, directly or indirectly, to tissue damage. Consequently, inhibiting the recruitment of these cells is beneficial in a wide range of diseases.

CCR5 is also a co-receptor for HIV-1 and other viruses, allowing these viruses to enter cells. Blocking the receptor with a CCR5 antagonist or inducing receptor internalisation with a CCR5 agonist protects cells from viral infection.

The present invention provides a compound of formula (I):

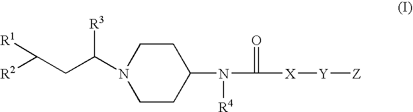

wherein
R¹ is C₁₋₈ alkyl, C(O)NR¹⁰R¹¹, C(O)₂R¹², NR¹³C(O)R¹⁴, NR¹⁵C(O)NR¹⁶R¹⁷, NR¹⁸C(O)₂R¹⁹, heterocyclyl, aryl or heteroaryl;
R¹⁰, R¹³, R¹⁵, R¹⁶ and R¹⁸ are hydrogen or C₁₋₆ alkyl;
R¹¹, R¹², R¹⁴, R¹⁷ and R¹⁹ are C₁₋₈ alkyl (optionally substituted by halo, hydroxy, C₁₋₆ alkoxy, C₁₋₆ haloalkoxy, C₃₋₆ cycloalkyl (optionally substituted by halo), C₅₋₆ cycloalkenyl, S(C₁₋₄ alkyl), S(O)(C₁₋₄ alkyl), S(O)₂(C₁₋₄ alkyl), heteroaryl, aryl, heteroaryloxy or aryloxy), aryl, heteroaryl, C₃₋₇ cycloalkyl (optionally substituted by halo, C₁₋₄ alkyl or C₁₋₄ haloalkyl), C₄₋₇ cycloalkyl fused to a phenyl ring, C₅₋₇ cycloalkenyl, or heterocyclyl; or
R¹¹, R¹², R¹⁴ and R¹⁷ can also be hydrogen;
or R¹⁰ and R¹¹, and/or R¹⁶ and R¹⁷ may join to form a 4-, 5- or 6-membered ring which optionally includes a nitrogen, oxygen or sulphur atom, said ring being optionally substituted by C₁₋₆ alkyl, C₁₋₆ haloalkyl, S(O)ₗ(C₁₋₆ alkyl) or C(O)(C₁₋₆ alkyl);
R² is C₁₋₆ alkyl, phenyl, heteroaryl or C₃₋₇ cycloalkyl;
when X is NR⁵, Y is absent or is CH₂;
when X is CH₂, Y is absent, CH₂, NR⁶, O, S, S(O) or S(O)₂;
Z is a 5- or 6-membered heterocyclyl ring;
R³, R⁵ and R⁶ are, independently, hydrogen or C₁₋₆ alkyl;
R⁴ is hydrogen, C₁₋₄ alkyl, C₃₋₄ alkenyl, C₃₋₄ alkynyl or C₃₋₆ cycloalkyl;
aryl, phenyl and heteroaryl moieties are independently optionally substituted by: halo, cyano, nitro, hydroxy, OC(O)NR²⁰R²¹, NR²²R²³, NR²⁴C(O)R²⁵, NR²⁶C(O)NR²⁷R²⁸, S(O)₂R²⁹R³⁰, NR³¹S(O)₂R³², C(O)NR³³R³⁴, CO₂R³⁶, NR³⁷CO₂R³⁸, S(O)_{q}R³⁹, OS(O)₂R⁴⁹, C₁₋₆ alkyl (optionally mono-substituted by S(O)₂R⁵⁰ or C(O)NR⁵¹R⁵²), C₂₋₆ alkenyl, C₂₋₆ alkynyl, C₃₋₁₀ cycloalkyl, C₁₋₆ haloalkyl, C₁₋₆ alkoxy(C₁₋₆)alkyl, C₁₋₆ alkoxy (optionally mono-substituted by CO₂R⁵³, C(O)NR⁵⁴R⁵⁵, cyano, heteroaryl or C(O)NHS(O)₂R⁵⁶), NHC(O)NHR⁵⁷, C₁₋₆ haloalkoxy, phenyl, phenyl(C₁₋₄)alkyl, phenoxy, phenylthio, phenylS(O), phenylS(O)₂, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃;
unless otherwise stated heterocyclyl moieties are independently optionally substituted by: C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)) or heteroaryl {which itself optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, heteroaryl {optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, S(O)₂NR⁴⁰R⁴¹, C(O)R⁴², C(O)₂(C₁₋₆ alkyl) (such as tert-butoxycarbonyl), C(O)₂(phenyl(C₁₋₂ alkyl)) (such as benzyloxycarbonyl), C(O)NHR⁴³, S(O)₂R⁴⁴, NHS(O)₂NHR⁴⁵, NHC(O)R⁴⁶, NHC(O)NHR⁴⁷ or NHS(O)₂R⁴⁸, provided none of these last four substituents is linked to a ring nitrogen;
k, l, p and q are, independently, 0, 1 or 2;
R²⁰, R²², R²⁴, R²⁶, R²⁷, R²⁹, R³¹, R³³R³⁷, R⁴⁰, R⁵¹ and R⁵⁴ are, independently, hydrogen or C₁₋₆ alkyl;
R²¹, R²³, R²⁵, R²⁸, R³⁰, R³², R³⁴, R³⁶, R³⁸, R³⁹, R⁴¹, R⁴², R⁴³, R⁴⁴, R⁴⁵, R⁴⁶, R⁴⁷, R⁴⁸, R⁴⁹, R⁵⁰, R⁵², R⁵³, R⁵⁵, R⁵⁶ and R⁵⁷ are, independently, C₁₋₆ alkyl (optionally substituted by halo, hydroxy, C₁₋₆ alkoxy, C₁₋₆ haloalkoxy, C₃₋₆ cycloalkyl, C₅₋₆ cycloalkenyl, S(C₁₋₄ alkyl), S(O)(C₁₋₄ alkyl), S(O)₂(C₁₋₄ alkyl), heteroaryl, phenyl, heteroaryloxy or phenyloxy), C₃₋₇ cycloalkyl, phenyl or heteroaryl; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
R²¹, R²³, R²⁵, R²⁸, R³⁰, R³⁴, R³⁵, R³⁶, R⁴¹, R⁴², R⁴³, R⁴⁵, R⁴⁶, R⁴⁷, R⁵², R⁵³, R⁵⁵ and R⁵⁷ may additionally be hydrogen;
alternatively, R²⁰ and R²¹, and/or R²² and R²³, and/or R²⁷ and R²⁸, and/or R²⁹ and R³⁰, and/or R³³ and R³⁴, and/or R⁵¹ and R⁵² and/or R⁵⁴ and R⁵⁵, and/or R⁴⁰ and R⁴¹ may join to form a 5- or 6-membered ring which is optionally substituted with halo, C₁₋₄ alkyl or phenyl (wherein the phenyl ring is optionally substituted by halo, cyano, nitro, hydroxy, C₁₋₄ alkyl, C₁₋₄ alkoxy, S(O)ₘC₁₋₄ alkyl, S(O)₂NH₂, S(O)₂NH(C₁₋₄ alkyl), S(O)₂N(C₁₋₄ alkyl)₂, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃);
m is 0, 1 or 2;
or a pharmaceutically acceptable salt thereof.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts such as a hydrochloride, hydrobromide, phosphate, acetate, fumarate, maleate, tartrate, citrate, oxalate, methanesulphonate, succinate or p-toluenesulphonate.

The compounds of the invention may exist as solvates (such as hydrates) and the present invention covers all such solvates.

Alkyl groups and moieties are straight or branched chain and are, for example, methyl, ethyl, n-propyl, iso-propyl, n-butyl, sec-butyl or tert-butyl. Methyl is sometimes abbreviated to Me hereinbelow.

Haloalkyl comprises, for example, one to six, such as one to three, halogen (such as fluorine) atoms, and is, for example, CF₃ or CH₂CF₃.

Cycloalkyl is, for example, cyclopropyl, cyclopentyl or cyclohexyl.

Phenyl(C₁₋₄ alkyl) is, for example, benzyl, 1-(phenyl)eth-1-yl or 1-(phenyl)eth-2-yl.

Heteroaryl(C₁₋₄ alkyl) is, for example, pyridinylmethyl, pyrimidinylmethyl or 1-(pyridinyl)eth-2-yl.

Phenyl(C₁₋₄ alkoxy) is, for example, benzyloxy or 1-(phenyl)eth-1-yloxy.

Aryloxy is, for example, phenoxy.

Heteroaryloxy is, for example, pyridinyloxy or pyrimidinyloxy.

Heteroaryl(C₁₋₄ alkoxy) is, for example, pyridinylmethoxy, pyrimidinylmethoxy or 1-(pyridinyl)eth-2-oxy.

Heteroaryl is an aromatic 5 or 6 membered ring, optionally fused to one or more other rings, comprising at least one heteroatom selected from the group comprising nitrogen, oxygen and sulphur; or an N-oxide thereof, or an S-oxide or S-dioxide thereof. Heteroaryl is, for example, furyl, thienyl (also known as thiophenyl), pyrrolyl, thiazolyl, isothiazolyl, pyrazolyl, oxazolyl, isoxazolyl, imidazolyl, [1,2,4]-triazolyl, tetrazolyl, pyridinyl, pyrimidinyl, pyrazinyl, indolyl, benzo[b]furyl (also known as benzfuryl), benz[b]thienyl (also known as benzthienyl or benzthiophenyl), indazolyl, benzimidazolyl, benztriazolyl, benzoxazolyl, benzthiazolyl, 1,2,3-benzothiadiazolyl, an imidazopyridinyl (such as imidazo[1,2a]pyridinyl), thieno[3,2-b]pyridin-6-yl, 1,2,3-benzoxadiazolyl (also known as benzo[1,2,3]thiadiazolyl), 2,1,3-benzothiadiazolyl, benzofurazan (also known as 2,1,3-benzoxadiazolyl), quinoxalinyl, a pyrazolopyridine (for example 1H-pyrazolo[3,4-b]pyridinyl), quinolinyl, isoquinolinyl, a naphthyridinyl (for example [1,6]naphthyridinyl or [1,8]naphthyridinyl), a benzothiazinyl or dibenzothiophenyl (also known as dibenzothienyl); or an N-oxide thereof, or an S-oxide or S-dioxide thereof.

Suitable 5- or 6-membered heterocyclyl rings (the group Z) include rings having one or two nitrogen atoms and, optionally, one oxygen or sulphur atom. Suitable rings are, for example, piperidine, piperazine, morpholine, thiomorpholine or pyrrolidine. In one aspect of the invention Z is piperidine, piperazine or pyrrolidine (such as piperidine or piperazine).

In a further aspect the present invention provides a compound of formula (I) wherein, unless specified otherwise, aryl, phenyl and heteroaryl moieties are independently optionally substituted by one or more of halo, hydroxy, nitro, S(C₁₋₆ alkyl), S(O)(C₁₋₆ alkyl), S(O)₂(C₁₋₆ alkyl), S(O)₂NH₂, S(O)₂NH(C₁₋₆ alkyl), S(O)₂N(C₁₋₆ alkyl)₂, cyano, C₁₋₆ alkyl, C₁₋₆ alkoxy, CH₂S(O)₂(C₁₋₆ alkyl), OS(O)₂(C₁₋₆ alkyl), OCH₂heteroaryl (such as OCH₂tetrazolyl), OCH₂CO₂H, OCH₂CO₂(C₁₋₆ alkyl), OCH₂C(O)NH₂, OCH₂C(O)NH(C₁₋₆ alkyl), OCH₂CN, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, C(O)[N-linked heterocyclyl], CO₂H, CO₂(C₁₋₆ alkyl), NHC(O)(C₁₋₆ alkyl), NHC(O)O(C₁₋₆ alkyl), NHS(O)₂(C₁₋₆ alkyl), CF₃, CHF₂, CH₂F, CH₂CF₃, OCF₃, phenyl, heteroaryl, phenyl(C₁₋₄ alkyl), heteroaryl(C₁₋₄ alkyl), NHC(O)phenyl, NHC(O)heteroaryl, NHC(O)(C₁₋₄ alkyl)phenyl, NHC(O)(C₁₋₄ alkyl)heteroaryl, NHS(O)₂phenyl, NHS(O)₂heteroaryl, NHS(O)₂(C₁₋₄ alkyl)phenyl, NHS(O)₂(C₁₋₄ alkyl)heteroaryl, NHC(O)NH(C₁₋₆ alkyl), NHC(O)NH(C₃₋₇ cycloalkyl), NHC(O)NHphenyl, NHC(O)NHheteroaryl, NHC(O)NH(C₁₋₄ alkyl)phenyl or NHC(O)NH(C₁₋₄ alkyl)heteroaryl; wherein the foregoing phenyl and heteroaryl groups are optionally substituted by halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃.

In another aspect the present invention provides a compound of formula (I) wherein, unless specified otherwise, aryl, phenyl and heteroaryl moieties are independently optionally substituted by one or more of halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), CF₃, CHF₂, CH₂F, CH₂CF₃ or OCF₃.

In another aspect of the invention R¹⁰, R¹³, R¹⁵, R¹⁶ and R¹⁵ are hydrogen or C₁₋₄ alkyl (for example methyl). In yet another aspect R¹⁰, R¹³, R¹⁵, R¹⁶ and R¹⁸ are hydrogen.

In a further aspect of the invention R¹¹, R¹², R¹⁴, R¹⁷, R¹⁸ and R¹⁹ are C₁₋₈alkyl (optionally substituted by halo, C₁₋₆ alkoxy, C₁₋₆ haloalkoxy, C₃₋₆ cycloalkyl (optionally substituted by halo), C₅₋₆ cycloalkenyl, S(O)₂(C₁₋₄ alkyl), heteroaryl, phenyl, heteroaryloxy or aryloxy (for example phenoxy)), phenyl, heteroaryl, C₃₋₇ cycloalkyl (optionally substituted by halo or C₁₋₄ alkyl), C₄₋₇ cycloalkyl fused to a phenyl ring, C₅₋₇ cycloalkenyl, or, heterocyclyl (itself optionally substituted by oxo, C(O)(C₁₋₆ alkyl), S(O)ₖ(C₁₋₄ alkyl), halo or C₁₋₄ alkyl); k is 0, 1 or 2; or R¹⁰ and R¹¹, and/or R¹⁶ and R¹⁷ may join to form a 4-, 5- or 6-membered ring which optionally includes a nitrogen, oxygen or sulphur atom, said ring being optionally substituted by C₁₋₆ alkyl or C(O)(C₁₋₆ alkyl).

In yet another aspect of the invention R¹¹, R¹², R¹⁴, R¹⁷ and R¹⁹ are C₁₋₈ alkyl (optionally substituted by halo (such as fluoro)), phenyl (optionally substituted as recited above), C₃₋₆ cycloalkyl (optionally substituted by halo (such as fluoro)) or C-linked nitrogen containing heterocyclyl (optionally substituted on the ring nitrogen).

In a further aspect R¹ is NHC(O)R¹⁴, phenyl or heterocyclyl, wherein R¹⁴ is as defined above, and phenyl and heterocyclyl are optionally substituted as described above.

In another aspect of the invention R¹ is NR¹³C(O)R¹⁴, wherein R¹³ and R¹⁴ are as defined above. For example R¹³ is hydrogen.

In yet another aspect of the invention R¹⁴ is C₁₋₈ alkyl (optionally substituted by halo (such as fluoro, for example to form CF₃CH₂)), phenyl (optionally substituted as recited above), C₃₋₆ cycloalkyl (optionally substituted by halo (such as fluoro, for example to form 1,1-difluorocyclohex-4-yl)) or C-linked nitrogen containing heterocyclyl (such as tetrahydropyran or piperidine, optionally substituted on the ring nitrogen).

In another aspect the present invention provides a compound of the invention wherein R¹⁴ is C₁₋₈ alkyl (optionally substituted by halo (such as fluoro, for example to form CF₃CH₂)), phenyl (optionally substituted by halo) or C₅₋₆ cycloalkyl (optionally substituted by halo (such as fluoro, for example to form 1,1-difluorocyclohex-4-yl)).

In a further aspect of the invention heterocyclyl of R¹ is optionally substituted (such as singly substituted for example on a ring nitrogen atom when present) by C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio or S(O)₂(C₁₋₄ alkyl)} or heteroaryl {which itself optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio or S(O)₂(C₁₋₄ alkyl)}, heteroaryl {optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio or S(O)₂(C₁₋₄ alkyl)}, S(O)₂NR⁴⁰R⁴¹, C(O)R⁴², C(O)NHR⁴³ or S(O)₂R⁴⁴; wherein R⁴⁰, R⁴¹, R⁴², R⁴³ and R⁴⁴ are, independently, hydrogen or C₁₋₆ alkyl.

In yet another aspect of the invention R¹ is optionally substituted aryl (such as optionally substituted phenyl) or optionally substituted heteroaryl, wherein the optional substituents are as recited above.

In a further aspect of the invention when R¹ is heterocyclyl it is, for example, tetrahydropyran, tetrahydrothiopyran, tetrahydrodioxythiopyran, piperidine, piperazine, pyrrolidine or azetidine. In another aspect when R¹ is heterocyclyl it is, for example, piperidine, piperazine, pyrrolidine or azetidine.

In a further aspect of the invention R¹ is optionally substituted heterocyclyl, such as optionally substituted: piperidin-1-yl, piperidin-4-yl, piperazin-1-yl, pyrrolidin-1-yl, pyrrolidin-3-yl, azetidin-1-yl or azetidin-3-yl.

In a still further aspect of the invention the heterocyclyl of R¹ is mono-substituted by C₁₋₆ alkyl, C₃₋₇ cycloalkyl, phenyl {optionally substituted by halo (for example fluoro), C₁₋₄ alkyl (for example methyl), C₁₋₄ alkoxy (for example methoxy), CF₃ or OCF₃}, S(O)₂(C₁₋₄ alkyl) (for example S(O)₂CH₃, S(O)₂CH₂CH₃ or S(O)₂CH(CH₃)₂), S(O)₂(C₁₋₄ fluoroalkyl) (for example S(O)₂CF₃ or S(O)₂CH₂CF₃), S(O)₂phenyl {optionally substituted (such as mono-substituted) by halo (for example chloro), cyano, C₁₋₄ alkyl, C₁₋₄ alkoxy, CF₃, OCF₃, S(O)₂(C₁₋₄ alkyl) (for example S(O)₂CH₃ or S(O)₂CH₂CH₂CH₃) or S(O)₂(C₁₋₄ fluoroalkyl) (for example S(O)₂CH₂CF₃)}, benzyl {optionally substituted by halo (for example chloro or fluoro), C₁₋₄ alkyl, C₁₋₄ alkoxy (for example methoxy), CF₃ or OCF₃}, C(O)H, C(O)(C₁₋₄ alkyl), benzoyl {optionally substituted by halo (for example chloro or fluoro), C₁₋₄ alkyl (for example methyl), C₁₋₄ alkoxy, CF₃ or OCF₃}, C(O)₂(C₁₋₄ alkyl), C(O)NH₂, C(O)NH(C₁₋₄ alkyl) or C(O)NHphenyl {optionally substituted by halo (for example fluoro), C₁₋₄ alkyl, C₁₋₄ alkoxy, CF₃ or OCF₃}. Said heterocyclyl can also be mono-substituted by S(O)₂N(C₁₋₄ alkyl)₂. In a still further aspect when said heterocyclyl is a 4-substituted piperidin-1-yl, a 1-substituted piperidin-4-yl, a 4-substituted piperazin-1-yl, a 3-substituted pyrrolidin-1-yl, a 1-substituted pyrrolidin-3-yl, a 3-substituted azetidin-1-yl or a 1-substituted azetidin-3-yl (for example where said substituent is as recited earlier in this paragraph). In another aspect said heterocyclyl is a 1-substituted piperidin-4-yl or a 4-substituted piperazin-1-yl, wherein the substituent is S(O)₂(C₁₋₄ alkyl), S(O)₂(C₁₋₄ haloalkyl), S(O)₂(phenyl), S(O)₂N(C₁₋₄ alkyl)₂ or phenyl.

In another aspect of the invention R¹ is piperidinyl or piperazinyl (such as piperidin-4-yl or piperazin-1-yl), either of which is N-substituted by phenyl, S(O)₂R³⁹ (wherein R³⁹ is C₁₋₄ alkyl (such as methyl or ethyl), phenyl or CF₃) or S(O)₂NR²⁹R³⁰ (wherein R²⁹ and R³⁰ are, independently, C₁₋₄ alkyl (such as methyl)).

In yet another aspect of the invention R¹ is NHC(O)R¹⁴ wherein R¹⁴ is C₁₋₄ haloalkyl (for example C₁₋₄ fluoroalkyl, such as CH₂CF₃ or CH₂CH₂CF₃), phenyl (optionally substituted by halo) or C₃₋₆ cycloalkyl (substituted by one or two fluoros).

In a further aspect of the invention R¹ is phenyl optionally substituted by S(O)₂R³⁹ (wherein R³⁹ is C₁₋₄ alkyl (such as methyl)).

In a still further aspect of the invention R¹ is heteroaryl (such as pyridinyl) optionally substituted by CF₃.

In another aspect of the invention R¹ is heterocyclyl (such as tetrahydropyran or tetrahydrothiopyran).

In yet another aspect of the invention R² is phenyl or heteroaryl, either of which is optionally substituted by halo, C₁₋₄ alkyl, C₁₋₄ alkoxy, S(O)ₙ(C₁₋₄ alkyl), nitro, cyano or CF₃; wherein n is 0, 1 or 2, for example 0 or 2. When R² is heteroaryl it is, for example, an optionally substituted thiophenyl (that is, thienyl).

In a further aspect R² is phenyl or thienyl, either of which is optionally substituted by halo (such as chloro or fluoro) or CF₃.

In a still further aspect R² is phenyl optionally substituted by halo (such as fluoro) or CF₃. For example R² is phenyl, 3-fluorophenyl, 3-chlorophenyl, 3-CF₃-phenyl, 3,5-dichlorophenyl or 3,5-difluorophenyl. In a still further aspect of the invention R² is phenyl, 3-fluorophenyl or 3,5-difluorophenyl.

In another aspect of the invention R³ is hydrogen or methyl. In a further aspect of the invention when R³ is C₁₋₄ alkyl (such as methyl) the carbon to which R³ is attached has the R absolute configuration. In yet another aspect of the invention R³ is hydrogen.

In a further aspect of the invention R⁴ is hydrogen, methyl, ethyl, n-propyl, allyl or cyclopropyl. In another aspect R⁴ is ethyl.

In another aspect X is CH₂ and Y is absent.

In yet another aspect X is NH and Y is CH₂.

In a further aspect Z is heterocyclyl (such as piperidinyl or piperazinyl) optionally substituted (such as on a ring nitrogen) by C(O)(C₁₋₆ alkyl), C(O)(C₁₋₆ alkoxy) or S(O)₂(C₁₋₄ alkyl).

In another aspect the present invention provides a compound of formula (Ia):

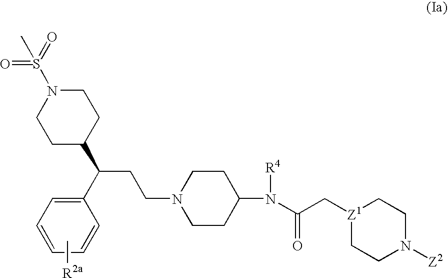

wherein
R^{2a} is one or 2 halogens (for example two fluoros); R⁴ is C₁₋₄ alkyl (for example ethyl or n-propyl); Z¹ is CH or N; and Z² is C(O)(C₁₋₆ alkyl) (such as acetyl), C(O)(C₁₋₆ alkoxy) (such as tert-butoxycarbonyl) or S(O)₂(C₁₋₄ alkyl) (such as S(O)₂CH₃).

In yet another aspect the present invention provides a compound of formula (Ib):

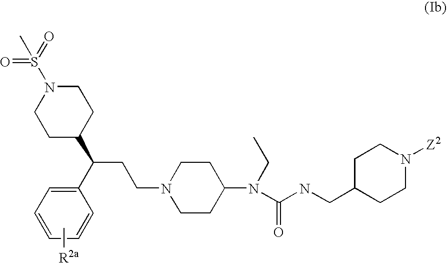

wherein R^{2a} and Z² are as defined above.

In yet another aspect the present invention provides a compound of formula (Ic):

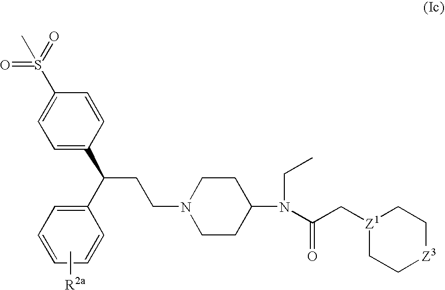

wherein R^{2a} and Z¹ are as defined above; and Z³ is oxygen or N—S(O)₂(C₁₋₄ alkyl) (such as N—S(O)₂CH₃).

In yet another aspect the present invention provides a compound of formula (Id):

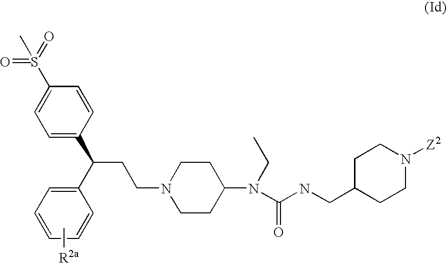

wherein R^{2a} and Z² are as defined above.

In yet another aspect the present invention provides a compound of formula (Ie):

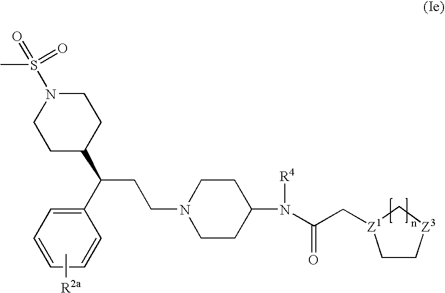

wherein R^{2a}, R⁴, Z¹ and Z³ are as defined above.

In yet another aspect the present invention provides a compound of formula (If):

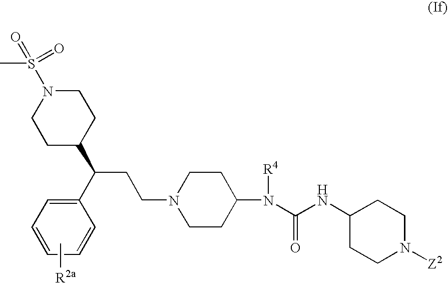

wherein R^{2a}, R⁴ and Z² are as defined above.

The compounds listed in Tables I, II, III IV, V and VI illustrate the invention.

TABLE I
Table I comprises compounds of formula (Ia).

| | |
|---|---|
| | (Ia) |
| 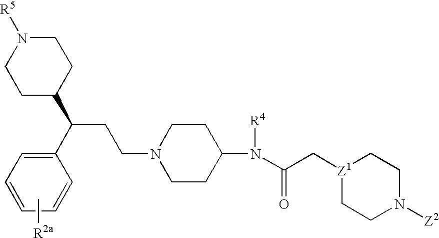 | |

| Compound | | | | | |
|---|---|---|---|---|---|
| No | R^{2a} | R⁴ | R⁵ | Z¹ | Z² |
| 1 | 3,5-difluoro | ethyl | SO₂Me | N | Tert-butoxycarbonyl |
| 2 | 3,5-difluoro | ethyl | SO₂Me | CH | Tert-butoxycarbonyl |
| 3 | 3,5-difluoro | ethyl | SO₂Me | CH | Hydrogen |
| 4 | 3,5-difluoro | ethyl | SO₂Me | N | Hydrogen |
| 5 | 3,5-difluoro | ethyl | SO₂Me | N | methanesulphonyl |
| 6 | 3,5-difluoro | ethyl | SO₂Me | CH | methanesulphonyl |
| 7 | 3,5-difluoro | allyl | SO₂Me | CH | methanesulphonyl |
| 8 | 3,5-difluoro | n-propyl | SO₂Me | CH | methanesulphonyl |
| 9 | 3,5-difluoro | ethyl | SO₂Me | N | acetyl |
| 10 | 3,5-difluoro | ethyl | SO₂Me | CH | acetyl |
| 11 | 3,5-difluoro | ethyl | SO₂CF₃ | CH | methanesulphonyl |
| 12 | 3,5-difluoro | CH₂CHMe₂ | SO₂Me | N | tert-butoxycarbonyl |
| 13 | 3,5-difluoro | CH₂CHMe₂ | SO₂Me | N | Hydrogen |
| 14 | 3,5-difluoro | CH₂CHMe₂ | SO₂Me | N | methanesulphonyl |
| 15 | 3,5-difluoro | CH₂CHMe₂ | SO₂Me | N | acetyl |
| 16 | 3-fluoro | CH₂CHMe₂ | SO₂Me | N | Hydrogen |
| 17 | 3-fluoro | CH₂CHMe₂ | SO₂Me | N | methanesulphonyl |
| 18 | 3-fluoro | CH₂CHMe₂ | SO₂Me | N | methoxycarbonyl |
| 19 | 3-fluoro | CH₂CHMe₂ | SO₂Me | N | acetyl |
| 20 | 3,5-difluoro | methyl | SO₂Me | N | tert-butoxycarbonyl |
| 21 | 3,5-difluoro | CH₂CHMe₂ | SO₂Me | N | methoxyethyl |
| 22 | 3,5-difluoro | CH₂-cyclopropyl | SO₂Me | N | methanesulphonyl |
| 23 | 3,5-difluoro | CH₂-cyclopropyl | SO₂Me | N | acetyl |
| 24 | 3,5-difluoro | methyl | SO₂Me | N | methanesulphonyl |
| 25 | 3,5-difluoro | methyl | SO₂Me | N | methoxycarbonyl |
| 26 | 3,5-difluoro | methyl | SO₂Me | N | acetyl |
| 27 | 3,5-difluoro | CH₂CHMe₂ | SO₂Me | N | methoxycarbonyl |
| 28 | 3,5-difluoro | CH₂-cyclopropyl | SO₂Me | N | methoxycarbonyl |
| 29 | 3,5-difluoro | CH₂CHMe₂ | SO₂Me | CH | methanesulphonyl |
| 30 | 3,5-difluoro | methyl | SO₂Me | CH | methanesulphonyl |

TABLE II
Table II comprises compounds of formula (Ib).

| | |
|---|---|
| | (Ib) |
| 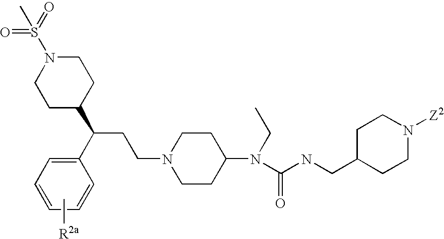 | |

| Compound No | R^{2a} | Z² |
|---|---|---|
| 1 | 3,5-difluoro | methanesulphonyl |

TABLE III
Table III comprises compounds of formula (Ic).

| | |
|---|---|
| | (Ic) |
| 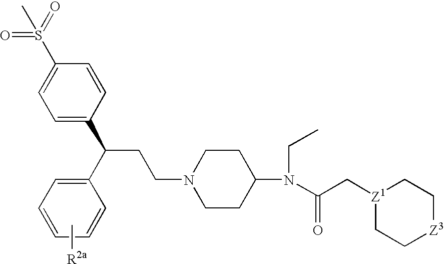 | |

| Compound | | | |
|---|---|---|---|
| No | R^{2a} | Z¹ | Z³ |
| 1 | 3,5-difluoro | CH | N—S(O)₂-methyl |
| 2 | 3,5-difluoro | CH | O |
| 3 | 3,5-difluoro | N | O |

TABLE IV
Table IV comprises compounds of formula (Id).

| | |
|---|---|
| | (Id) |
| 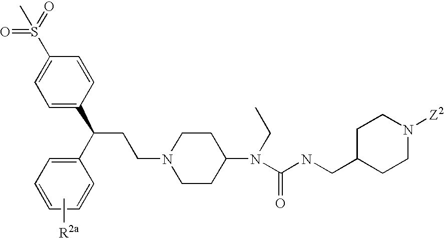 | |

| Compound No | R^{2a} | Z² |
|---|---|---|
| 1 | 3,5-difluoro | methanesulphonyl |

TABLE V
Table V comprises compounds of formula (1e)

| | |
|---|---|
| | (Ie) |
| 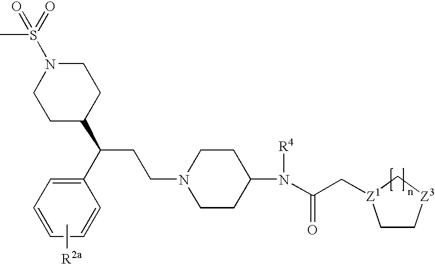 | |

| Compound No | R^{2a} | R⁴ | n | Z¹ | Z³ |
|---|---|---|---|---|---|
| 1 | 3,5-difluoro | CH₂CHMe₂ | 2 | C | O |
| 2 | 3,5-difluoro | CH₂CHMe₂ | 1 | C | O |
| 3 | 3,5-difluoro | CH₂CHMe₂ | 2 | N | SO₂ |
| 4 | 3,5-difluoro | CH₂CHMe₂ | 1 | C | NSO₂Me |
| 5 | 3,5-difluoro | ethyl | 1 | C | NSO₂Me |

TABLE VI
Table VI comprises compounds of formula (1f)

| | |
|---|---|
| | (If) |
| 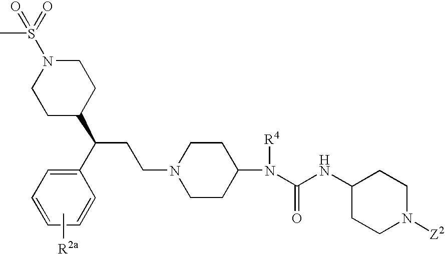 | |

| Compound No | R^{2a} | R⁴ | Z² |
|---|---|---|---|
| 1 | 3,5-difluoro | CH₂CHMe₂ | methanesulphonyl |
| 2 | 3,5-difluoro | CH₂CHMe₂ | methoxycarbonyl |
| 3 | 3,5-difluoro | CH₂CHMe₂ | acetyl |
| 4 | 3,5-difluoro | methyl | methanesulphonyl |
| 5 | 3,5-difluoro | methyl | methoxycarbonyl |

In yet another aspect the invention provides each individual compound listed in the tables above.

The compounds of formulae (I), (Ia), (Ib), (Ic), (Id), (Ie) and (If) can be prepared as shown below.

The compounds of the invention can be prepared by reacting a compound of formula (II):

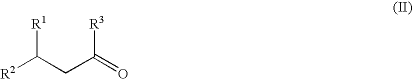

with a compound of formula (III):

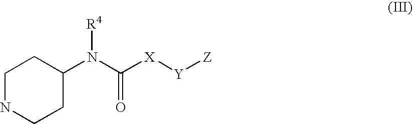

under reductive amination conditions {for example in the presence of a suitable solvent (such as an aliphatic alcohol such as methanol), a suitable organic acid (such as an aliphatic acid, for example acetic acid) and a suitable reducing agent (such as sodium triacetoxyborohydride or sodium cyanoborohydride)}.

Alternatively, a compound of the invention can be prepared by reacting a compound of formula (IV):

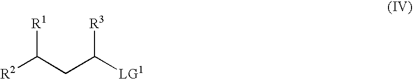

wherein the leaving group LG¹ is, for example, tosylate, mesylate, triflate or halogen; with a compound of formula (III), under standard literature conditions.

Alternatively, a compound of the invention can be prepared by reacting (V):

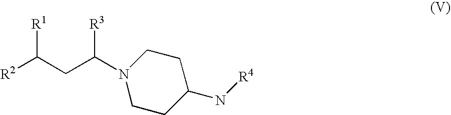

with:
when X is CH₂, a compound of formula (VI):

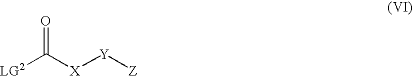

wherein LG² is, for example, halogen, an active ester or OH (thus forming a carboxylic acid), activated with a carbodiimide coupling agent such as HATU or the activated product of the reaction of an acid with carbonyldiimidazole; the reaction being carried out in an inert solvent (such as dichloromethane) in the presence of a base (such as triethylamine);

### OR

when X is NH, a compound of formula (VII):

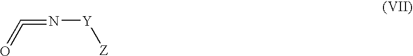

the reaction being carried out in an inert solvent (such as dichloromethane) in the presence of a base (such as triethylamine);

### OR

when X is NR⁵, a compound of formula (VIII):

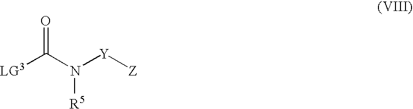

wherein LG³ is halogen or an active ester; the reaction being carried out in an inert solvent (such as dichloromethane) in the presence of a base (such as triethylamine).

A compound of formula (V) can be prepared by deprotecting (IX):

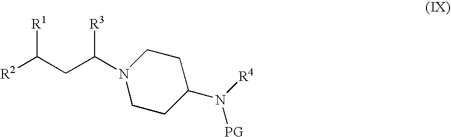

wherein the protecting group PG is, for example, benzyl, Cbz (benzyloxycarbonyl) or tert-butoxycarbonyl, and can be removed by hydrogenation or by treatment with acid (such as trifluoroacetic acid).

A compound of formula (IX) can be prepared by reacting (X):

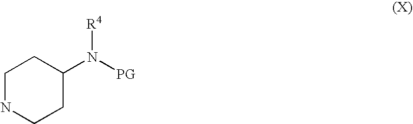

with a compound of formula (II) or (IV) above, employing the conditions outlined above for reacting a compound of formula (II) or (IV) with a compound of formula (III).

A compound of the invention wherein Y is absent, X is CH₂ and Z is a N containing heterocycle can be prepared by reacting a compound of formula (XI):

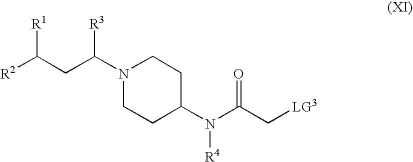

wherein LG³ is halogen (such as bromine), tosylate or mesylate), with an N-containing heterocycle in an inert solvent (such as dichloromethane, dioxane or tetrahydrofuran) in presence of base (such as triethylamine or diisopropylethylamine) at a temperature in the range from ambient to the boiling point of the solvent).

A compound of formula (XI) can be prepared by reaction of a compound of formula (V) with a halo acetic acid, using an acid coupling reagent known in the art, or with a haloacetyl halide.

In a still further aspect the invention provides processes for preparing the compounds of formulae (I), (Ia), (Ib), (Ic) (Id), (Ie) and (If). Many of the intermediates in the processes are novel and these are provided as further features of the invention.

The compounds of the invention have activity as pharmaceuticals, in particular as modulators (such as agonists, partial agonists, inverse agonists or antagonists) of chemokine receptor (especially CCR5) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

The compounds of the present invention are also of value in inhibiting the entry of viruses (such as human immunodeficiency virus (HIV)) into target calls and, therefore, are of value in the prevention of infection by viruses (such as HIV), the treatment of infection by viruses (such as HIV) and the prevention and/or treatment of acquired immune deficiency syndrome (AIDS).

According to a further feature of the invention there is provided a compound of the formula (I), (Ia), (Ib), (Ic), (Id) (Ie) or (If), or a pharmaceutically acceptable salt thereof, for use in a method of treatment of a warm blooded animal (such as man) by therapy (including prophylaxis).

According to a further feature of the present invention there is provided a method for modulating chemokine receptor activity (especially CCR5 receptor activity) in a warm blooded animal, such as man, in need of such treatment, which comprises administering to said animal an effective amount of a compound of the present invention, or a pharmaceutically acceptable salt thereof.

The present invention also provides the use of a compound of the formula (I), (Ia), (Ib), (Ic) (Id), (Ie) or (If) or a pharmaceutically acceptable salt thereof, as a medicament, especially a medicament for the treatment of transplant rejection, respiratory disease, psoriasis or rheumatoid arthritis (for example rheumatoid arthritis). [Respiratory disease is, for example, COPD, asthma {such as bronchial, allergic, intrinsic, extrinsic or dust asthma, particularly chronic or inveterate asthma (for example late asthma or airways hyper-responsiveness)} or rhinitis {acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis}; and is particularly asthma or rhinitis].

In another aspect the present invention provides the use of a compound of the formula (I), (Ia), (Ib), (Ic) (Id), (Ie) or (If) or a pharmaceutically acceptable salt thereof, in the manufacture of a medicament for use in therapy (for example modulating chemokine receptor activity (especially CCR5 receptor activity (especially rheumatoid arthritis)) in a warm blooded animal, such as man).

The invention also provides a compound of the formula (I), (Ia), (Ib), (Ic) (Id), (Ie) or (If) or a pharmaceutically acceptable salt thereof, for use as a medicament, especially a medicament for the treatment of rheumatoid arthritis.

In another aspect the present invention provides the use of a compound of the formula (I), (Ia), (Ib), (Ic) (Id), (Ie) or (If) or a pharmaceutically acceptable salt thereof, in the manufacture of a medicament for use in therapy (for example modulating chemokine receptor activity (especially CCR5 receptor activity (especially rheumatoid arthritis)) in a warm blooded animal, such as man).

The invention further provides the use of a compound of formula (I), (Ia), (Ib), (Ic) (Id), (Ie) or (If) or a pharmaceutically acceptable salt thereof, in the manufacture of a medicament for use in the treatment of:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;
(2) (bone and joints) arthrides including rheumatic, infectious, autoimmune, seronegative spondyloarthropathies (such as ankylosing spondylitis, psoriatic arthritis or Reiter's disease), Behçet's disease, Sjogren's syndrome or systemic sclerosis;
(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia greata or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allograft rejection) acute and chronic following, for example, transplantation of kidney, heart, liver, lung, bone marrow, skin or cornea; or chronic graft versus host disease; and/or
(6) (other tissues or diseases) Alzheimer's disease, multiple sclerosis, atherosclerosis, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura or disorders of the menstrual cycle;
   in a warm blooded animal, such as man.

The present invention further provides a method of treating a chemokine mediated disease state (especially a CCR5 mediated disease state) in a warm blooded animal, such as man, which comprises administering to a mammal in need of such treatment an effective amount of a compound of formula (I), (Ia), (Ib), (Ic) (Id), (Ie) or (If) or a pharmaceutically acceptable salt thereof.

In order to use a compound of the invention, or a pharmaceutically acceptable salt thereof or solvate thereof, for the therapeutic treatment of a warm blooded animal, such as man, in particular modulating chemokine receptor (for example CCR5 receptor) activity, said ingredient is normally formulated in accordance with standard pharmaceutical practice as a pharmaceutical composition.

Therefore in another aspect the present invention provides a pharmaceutical composition which comprises a compound of the formula (I), (Ia), (Ib), (Ic) (Id), (Ie) or (If) or a pharmaceutically acceptable salt thereof (active ingredient), and a pharmaceutically acceptable adjuvant, diluent or carrier. In a further aspect the present invention provides a process for the preparation of said composition which comprises mixing active ingredient with a pharmaceutically acceptable adjuvant, diluent or carrier. Depending on the mode of administration, the pharmaceutical composition will comprise, for example, from 0.05 to 99% w (percent by weight), such as from 0.05 to 80% w, for example from 0.10 to 70% w (such as from 0.10 to 50% w), of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 0.1 mg and 1 g of active ingredient.

In another aspect a pharmaceutical composition of the invention is one suitable for intravenous, subcutaneous or intramuscular injection.

Each patient may receive, for example, an intravenous, subcutaneous or intramuscular dose of 0.01 mgkg⁻¹ to 100 mgkg⁻¹ of the compound, preferably in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily pa renteral dose, the composition being administered 1 to 4 times per day.

The following illustrate representative pharmaceutical dosage forms containing the compound of formula (I), (Ia), (Ib), (Ic), (Id), (Ie) or (If) or a pharmaceutically acceptable salt thereof (hereafter Compound X), for therapeutic or prophylactic use in humans:

mg/tablet

(a)

| | |
|---|---|
| Tablet I | |
| Compound X | 100 |
| Lactose Ph.Eur. | 179 |
| Croscarmellose sodium | 12.0 |
| Polyvinylpyrrolidone | 6 |
| Magnesium stearate | 3.0 |

(b)

| | |
|---|---|
| Tablet II | |
| Compound X | 50 |
| Lactose Ph.Eur. | 229 |
| Croscarmellose sodium | 12.0 |
| Polyvinylpyrrolidone | 6 |
| Magnesium stearate | 3.0 |

(c)

| | |
|---|---|
| Tablet III | |
| Compound X | 1.0 |
| Lactose Ph.Eur. | 92 |
| Croscarmellose sodium | 4.0 |
| Polyvinylpyrrolidone | 2.0 |
| Magnesium stearate | 1.0 |

(d)

| Capsule | mg/capsule |
|---|---|
| Compound X | 10 |
| Lactose Ph.Eur. | 389 |
| Croscarmellose sodium | 100 |
| Magnesium stearate | 1.0 |

(e)

| Injection I | (50 mg/ml) |
|---|---|
| Compound X | 5.0% w/v |
| Isotonic aqueous solution | to 100% |

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. The tablets (a)-(c) may be enteric coated by conventional means, for example to provide a coating of cellulose acetate phthalate.

The invention further relates to combination therapies or compositions wherein a compound of formula (I), (Ia), (Ib), (Ic) (Id), (Ie) or (If) or a pharmaceutically acceptable salt thereof, or a pharmaceutical composition comprising a compound of formula (I), (Ia), (Ib), (Ic) (Id), (Ie) or (If) or a pharmaceutically acceptable salt thereof, is administered concurrently (possibly in the same composition) or sequentially with an agent for the treatment of any one of the above disease states.

In particular, for the treatment of the inflammatory diseases rheumatoid arthritis, psoriasis, inflammatory bowel disease, COPD, asthma and allergic rhinitis a compound of the invention can be combined with a TNF-α inhibitor (such as an anti-TNF monoclonal antibodie (such as Remicade, CDP-870 and D.sub2.E.sub7.), or a TNF receptor immunoglobulin molecule (such as Enbrel.reg.)), a non-selective COX-1/COX-2 inhibitor (such as piroxicam or diclofenac; a propionic acid such as naproxen, flubiprofen, fenoprofen, ketoprofen or ibuprofen; a fenamate such as mefenamic acid, indomethacin, sulindac or apazone; a pyrazolone such as phenylbutazone; or a salicylate such as aspirin), a COX-2 inhibitor (such as meloxicam, celecoxib, rofecoxib, valdecoxib or etoricoxib) low dose methotrexate, lefunomide; ciclesonide; hydroxychloroquine, d-penicillamine or auranofin, or parenteral or oral gold.

The present invention still further relates to the combination of a compound of the invention together with:
a leukotriene biosynthesis inhibitor, a 5-lipoxygenase (5-LO) inhibitor or a 5-lipoxygenase activating protein (FLAP) antagonist, such as zileuton, ABT-761, fenleuton, tepoxalin, Abbott-79175, Abbott-85761, an N-(5-substituted)-thiophene-2-alkylsulfonamide, a 2,6-di-tert-butylphenol hydrazones, a methoxytetrahydropyran such as Zeneca ZD-2138, SB-210661, a pyridinyl-substituted 2-cyanonaphthalene compound such as L-739,010; a 2-cyanoquinoline compound such as L-746,530; an indole or quinoline compound such as MK-591, MK-886 or BAY x 1005;
a receptor antagonist for a leukotriene LTB.sub4., LTC.sub4., LTD.sub4. or LTE.sub4. selected from the group consisting of a phenothiazin-3-one such as L-651,392; an amidino compound such as CGS-25019c; a benzoxalamine such as ontazolast; a benzenecarboximidamide such as BIIL 284/260; or a compound such as zafirlukast, ablukast, montelukast, pranlukast, verlukast (MK-679), RG-12525, Ro-245913, iralukast (CGP 45715A) or BAY x 7195;
a PDE4 inhibitor including an inhibitor of the isoform PDE4D;
an antihistaminic H.sub1. receptor antagonist such as cetirizine, loratadine, desloratadine, fexofenadine, astemizole, azelastine or chlorpheniramine;
a gastroprotective H.sub2. receptor antagonist;
an α.sub1.- and α.sub2.-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride or ethylnorepinephrine hydrochloride;
an anticholinergic agent such as ipratropium bromide, tiotropium bromide, oxitropium bromide, pirenzepine or telenzepine;
a β.sub1.- to β.sub4.-adrenoceptor agonist such as metaproterenol, isoproterenol, isoprenaline, albuterol, salbutamol, formoterol, salmeterol, terbutaline, orciprenaline, bitolterol mesylate or pirbuterol, or a methylxanthanine including theophylline and aminophylline; sodium cromoglycate; or a muscarinic receptor (M1, M2, and M3) antagonist;
an insulin-like growth factor type I (IGF-1) mimetic;
an inhaled glucocorticoid with reduced systemic side effects, such as prednisone, prednisolone, flunisolide, triamcinolone acetonide, beclomethasone dipropionate, budesonide, fluticasone propionate or mometasone furoate;
an inhibitor of a matrix metalloprotease (MMP), such as a stromelysin, a collagenase, or a gelatinase or aggrecanase; such as collagenase-1 (MMP-1), collagenase-2 (MMP-8), collagenase-3 (MMP-13), stromelysin-1 (MMP-3), stromelysin-2 (MMP-10), and stromelysin-3 (MMP-11) or MMP-12;
a modulator of chemokine receptor function such as CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family;
an osteoporosis agent such as roloxifene, droloxifene, lasofoxifene or fosomax;
an immunosuppressant agent such as FK-506, rapamycin, cyclosporine, azathioprine or methotrexate; or,
an existing therapeutic agent for the treatment of osteoarthritis, for example a non-steroidal anti-inflammatory agent (hereinafter NSAID's) such as piroxicam or diclofenac, a propionic acid such as naproxen, flubiprofen, fenoprofen, ketoprofen or ibuprofen, a fenamate such as mefenamic acid, indomethacin, sulindac or apazone, a pyrazolone such as phenylbutazone, a salicylate such as aspirin, a COX-2 inhibitor such as celecoxib, valdecoxib, rofecoxib or etoricoxib, an analgesic or intra-articular therapy such as a corticosteroid or a hyaluronic acid such as hyalgan or synvisc, or a P2X7 receptor antagonist.

The present invention still further relates to the combination of a compound of the invention together with: (i) a tryptase inhibitor; (ii) a platelet activating factor (PAF) antagonist; (iii) an interleukin converting enzyme (ICE) inhibitor; (iv) an IMPDH inhibitor; (v) an adhesion molecule inhibitor including a VLA-4 antagonist; (vi) a cathepsin; (vii) a MAP kinase inhibitor; (viii) a glucose-6 phosphate dehydrogenase inhibitor; (ix) a kinin-B.sub1.- and B.sub2.-receptor antagonist; (x) an anti-gout agent, e.g., colchicine; (xi) a xanthine oxidase inhibitor, e.g., allopurinol; (xii) an uricosuric agent, e.g., probenecid, sulfinpyrazone or benzbromarone; (xiii) a growth hormone secretagogue; (xiv) a transforming growth factor (TGFβ); (xv) a platelet-derived growth factor (PDGF); (xvi) a fibroblast growth factor, e.g., basic fibroblast growth factor (bFGF); (xvii) a granulocyte macrophage colony stimulating factor (GM-CSF); (xviii) a capsaicin cream; (xix) a Tachykinin NK.sub1. and NK.sub3. receptor antagonist selected from the group consisting of NKP-608C; SB-233412 (talnetant); and D-4418; (xx) an elastase inhibitors selected from the group consisting of UT-77 and ZD-0892; (xxi) a TNFα converting enzyme inhibitor (TACE); (xxii) an induced nitric oxide synthase inhibitor (iNOS); or (xxiii) a chemoattractant receptor-homologous molecule expressed on TH2 cells (a CRTH2 antagonist).

The invention will now be illustrated by the following non-limiting Examples in which, unless stated otherwise:

(i) temperatures are given in degrees Celsius (° C.); operations were carried out at room or ambient temperature, that is, at a temperature in the range of 18-25° C.;
(ii) organic solutions were dried over anhydrous magnesium sulfate; evaporation of solvent was carried out using a rotary evaporator under reduced pressure (600-4000 Pascals; 4.5-30 mm Hg) with a bath temperature of up to 60° C.;
(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Glamorgan, UK. Where “Argonaut™PS-tris-amine scavenger resin” is referred to, this means a tris-(2-aminoethyl)amine polystyrene resin obtained from Argonaut Technologies Inc., 887 Industrial Road, Suite G, San Carlos, Calif., USA.
(iv) in general, the course of reactions was followed by TLC and reaction times are given for illustration only;
(v) yields, when given, are for illustration only and are not necessarily those which can be obtained by diligent process development; preparations were repeated if more material was required;
(vi) when given, ¹H NMR data is quoted and is in the form of delta values for major diagnostic protons, given in parts per million (ppm) relative to tetramethylsilane (TMS) as an internal standard, determined at 400 MHz using perdeuterio DMSO (CD₃SOCD₃) as the solvent unless otherwise stated; coupling constants (J) are given in Hz;
(vii) chemical symbols have their usual meanings; SI units and symbols are used;
(viii) solvent ratios are given in percentage by volume;
(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+H)⁺;
(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion—(M+H)⁺ and
(xi) the following abbreviations are used:
DMSO dimethyl sulfoxide;
DMF N-dimethylformamide;
DCM dichloromethane;
THF tetrahydrofuran;
DIPEA N,N-diisopropylethylamine;
NMP N-methylpyrrolidinone;
HATU O-(7-Azabenzotriazol-1-yl)-N,N,N′,N-tetramethyluronium hexafluorophosphate;
HBTU O-(7-Benzotriazol-1-yl)-N,N,N′,N′-tetramethyluronium hexafluorophosphate;
Boc tert-butoxycarbonyl
MeOH methanol;
EtOH ethanol;
EtOAc ethyl acetate;
MP macro porous; and,
PS polymer supported.

### EXAMPLE 1

This Example illustrates the preparation of tert-butyl 4-{2-[(1-{(3R)-3-(3,5-difluorophenyl)-3-[1-(methylsulfonyl)piperidin-4-yl]propyl}piperidin-4-yl)(ethyl)amino]-2-oxoethyl}piperazine-1-carboxylate (Compound 1 Table I).

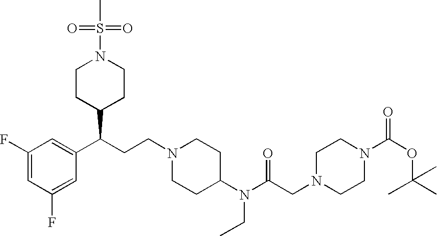

MP-triacetoxyborohydride (2.5 g) was added to a solution of (3R)-3-(3,5-difluorophenyl)-3-[1-(methylsulfonyl)piperidin-4-yl]propanal (662 mg) (Method A) and tert-butyl 4-{2-[ethyl(piperidin-4-yl)amino]-2-oxoethyl}piperazine-1-carboxylate (Method B) (704 mg) in dichloromethane (25 ml) and the mixture was stirred for 18 hours. The reaction mixture was filtered and the resin was washed with a 1:9 mixture of methanol in dichloromethane (50 ml). The combined filtrates were evaporated to dryness and the residue was purified by passing down a 40 g silica column eluted with a solvent gradient of ethyl acetate to 30% methanol-ethyl acetate. The title compound was obtained in 58% yield, LC-MS M+H=670.

¹H NMR: 1.15 (3H, m), 1.20-2.17 (14H, m), 1.42 (9H, s), 2.36-2.54 (6H, m), 2.62 (2H, t), 2.74 (3H, s), 2.90 (2H, m), 3.18 (2H, d), 3.22-3.48 (6H, m), 3.71 (1H, d), 3.79 & 4.29 (1H, m), 3.85 (1H, d), 6.63 (3H, m).

### EXAMPLE 1b

In an analogous manner but using tert-butyl 4-{2-[isobutyl(piperidin-4-yl)amino]-2-oxoethyl}piperazine-1-carboxylate (Method B) instead of tert-butyl 4-{2-[ethyl(piperidin-4-yl)amino]-2-oxoethyl}piperazine-1-carboxylate was prepared tert-butyl 4-{2-[(1-{(3R)-3-(3,5-difluorophenyl)-3-[1-(methylsulfonyl)piperidin-4-yl]propyl}piperidin-4-yl)(isobutyl)amino]-2-oxoethyl}piperazine-1-carboxylate (Compound 12 Table I), LC-MS M+H=698

NMR CDCl3 0.65 (d, 3H) 0.75 (d, 3H) 1.1-1.3 (m, 3H) 1.4 (s, 9H) 1.45-2.1 (m, 14H) 2.3-2.6 (m, 10H) 2.7 (s, 3H) 2.8-3.2 (m, 4H) 3.3-3.4 (m, 3H) 3.7 (m, 1H) 3.8 (m, 1H) 6.6 (m, 3H)

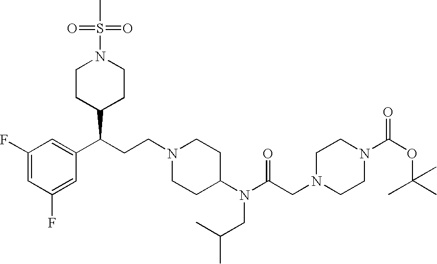

### EXAMPLE 1c

In an analogous manner but using tert-butyl 4-{2-[methyl(piperidin-4-yl)amino]-2-oxoethyl}piperazine-1-carboxylate (Method B) instead of tert-butyl 4-{2-[ethyl(piperidin-4-yl)amino]-2-oxoethyl}piperazine-1-carboxylate was prepared tert-butyl 4-{2-[(1-{(3R)-3-(3,5-difluorophenyl)-3-[1-(methylsulfonyl)piperidin-4-yl]propyl}piperidin-4-yl)(methyl)amino]-2-oxoethyl}piperazine-1-carboxylate (Compound 20 Table I); LC-MS M+H=656

NMR CDCl₃ 1.2-1.4 (2H, m) 1.45 (9H, s) 1.5-2.2 (14H, m) 2.35-2.5 (5H, m) 2.6 (1H, m) 2.7 (3H, s) 2.75-2.95 (5H, m) 3.2 (2H, d) 3.45-3.5 (4H, m) 3.7 (1H, m) 3.75, 4.4 (1H, m) 3.85 (1H, m) 6.7 (3H, m)

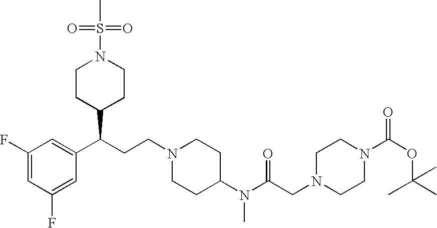

### EXAMPLE 1d

In an analogous manner but using tert-butyl 4-{2-[isobutyl(piperidin-4-yl)amino]-2-oxoethyl}piperazine-1-carboxylate (Method B) instead of tert-butyl 4-{2-[ethyl(piperidin-4-yl)amino]-2-oxoethyl}piperazine-1-carboxylate and (3R)-3-(3-fluorophenyl)-3-[1-(methylsulfonyl)piperidin-4-yl]propanal (Method A) was prepared tert-butyl 4-{2-[(1-{(3R)-3-(3,5-difluorophenyl)-3-[1-(methylsulfonyl)piperidin-4-yl]propyl}piperidin-4-yl)(isobutyl)amino]-2-oxoethyl}piperazine-1-carboxylate; LC-MS M+H=680

NMR CDCl₃ 0.75 (3H, d) 0.85 (3H, d) 1.1-1.35 (4H, m) 1.4 (9H, s) 1.45-2.1 (16H, m) 2.3-2.5 (5H, m) 2.55 (1H, t) 2.65 (3H, s) 2.7-3.05 (4H, m) 3.1 (1H, s) 3.3-3.4 (3H, m) 3.6 (1H, m) 3.8-4 (1H, m) 6.7-6.9 (3H, m) 7.2 (1H, m)

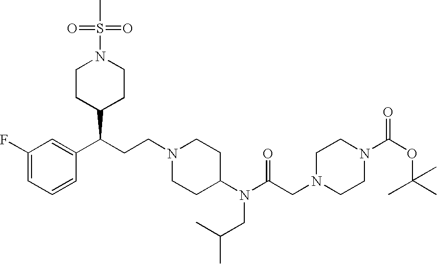

### EXAMPLE 2

This Example describes the preparation of tert-butyl 4-{2-[(1-{(3R)-3-(3,5-difluorophenyl)-3-[1-(methylsulfonyl)piperidin-4-yl]propyl}piperidin-4-yl)(ethyl)amino]-2-oxoethyl}piperidine-1-carboxylate (Compound 2 Table I).

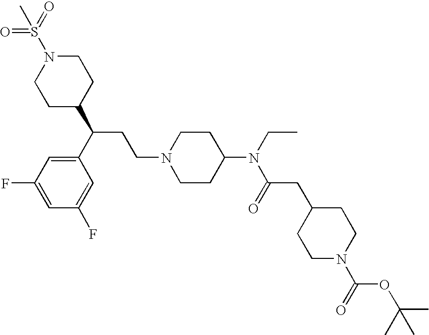

MP-triacetoxyborohydride (2.5 g) was added to a solution of (3R)-3-(3,5-difluorophenyl)-3-[1-(methylsulfonyl)piperidin-4-yl]propanal (662 mg) (Method A) and tert-butyl 4-{2-[ethyl(piperidin-4-yl)amino]-2-oxoethyl}piperidine-1-carboxylate (Method B) (706 mg) in dichloromethane (25 ml) and the mixture was stirred for 18 hours. The reaction mixture was filtered and the resin was washed with a 1:9 mixture of methanol in dichloromethane (50 ml). The combined filtrates were evaporated to dryness and the residue was purified by passing down a 40 g silica column eluted with a solvent gradient of ethyl acetate to 10% methanol-ethyl acetate to give the title compound yield, 684 mg, LC-MS M+H=669.

¹H NMR: 1.10-2.23 (25H, m), 1.43 (9H, s), 2.38 (1H, t), 2.51 (1H, t), 2.62 (1H, t), 2.72 (2H, m), 2.73 (3H, s), 2.88 (2H, m), 3.24 (2H, m), 3.48 & 4.38 (1H, m), 3.72 (1H, d), 3.84 (1H, d), 4.08 (2H, m), 6.62 (3H, m).

### EXAMPLE 3

This Example describes the preparation of N-(1-{(3R)-3-(3,5-difluorophenyl)-3-[1-(methylsulfonyl)piperidin-4-yl]propyl}piperidin-4-yl)-N-ethyl-2-piperidin-4-ylacetamide (Compound 3 Table I).

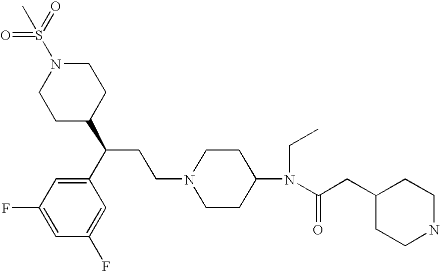

tert-Butyl 4-{2-[(1-{(3R)-3-(3,5-difluorophenyl)-3-[1-(methylsulfonyl)piperidin-4-yl]propyl}piperidin-4-yl)(ethyl)amino]-2-oxoethyl}piperidine-1-carboxylate (635 mg) was added to 4M HCl in dioxane (10 ml) and the mixture was allowed to stand for 15 minutes. Methanol (10 ml) was added and the solution was stirred for 45 minutes. The solvent was evaporated to give a white foam, yield 560 mg, LC-MS M+H 569.

¹H NMR (CDCl₃): 1.10-2.14 (23H, m), 2.22 (2H, m), 2.42 (1H, t), 2.54 (1H, t), 2.66 (3H, m), 2.74 (3H, s), 2.88 (2H, m), 3.12 (2H, d), 3.28 (2H, m), 3.50 & 4.38 (1H, m), 3.72 (1H, d), 3.84 (1H, d), 6.64 (3H, m)

Using tert-butyl 4-{2-[(1-{(3R)-3-(3,5-difluorophenyl)-3-[1-(methylsulfonyl)piperidin-4-yl]propyl}piperidin-4-yl)(ethyl)amino]-2-oxoethyl}piperazine-1-carboxylate (Compound 1 Table I) as starting material there is obtained N-(1-{(3R)-3-(3,5-difluorophenyl)-3-[1-(methylsulfonyl)piperidin-4-yl]propyl}piperidin-4-yl)-N-ethyl-2-piperazin-1-ylacetamide (Compound 4 Table I); LC-MS M+H 570, ¹H NMR (CDCl₃): 1.10-2.14 (18H, m), 2.36-2.56 (6H, m), 2.64 (2H, t), 2.74 (3H, s), 2.78-2.96 (5H, m), 3.14 (2H, d), 3.32 (2H, m), 3.72 (1H, m), 3.78 & 4.28 (1H, m), 3.86 (1H, m), 6.64 (3H, m).

### EXAMPLE 3a

Using tert-butyl 4-{2-[(1-{(3R)-3-(3,5-difluorophenyl)-3-[1-(methylsulfonyl)piperidin-4-yl]propyl}piperidin-4-yl)(isobutyl)amino]-2-oxoethyl}piperazine-1-carboxylate (Compound 12 Table I) as starting material there is obtained N-(1-{(3R)-3-(3,5-difluorophenyl)-3-[1-(methylsulfonyl)piperidin-4-yl]propyl}piperidin-4-yl)-N-isobutyl-2-piperazin-1-ylacetamide (Compound 13 Table I); LC-MS M+H 598

NMR CDCl₃ 0.7 (d, 3H) 0.8 (d, 3H) 1-2.1 (m, 20H) 2.3-2.45 (m, 5H) 2.6 (t, 1H) 2.7 (s, 3H) 2.75-2.8 (m, 2H) 2.9 (d, 1H) 3.1 (m, 2H) 3.7 (d, 1H) 3.8 (m, 1H) 6.6 (m, 3H)

### EXAMPLE 3b

Using tert-butyl 4-{2-[(1-{(3R)-3-(3,5-difluorophenyl)-3-[1-(methylsulfonyl)piperidin-4-yl]propyl}piperidin-4-yl)(isobutyl)amino]-2-oxoethyl}piperazine-1-carboxylate (Example 1d) as starting material was prepared N-(1-{(3R)-3-(3-fluorophenyl)-3-[1-(methylsulfonyl)piperidin-4-yl]propyl}piperidin-4-yl)-N-isobutyl-2-piperazin-1-ylacetamide (Compound 16 Table I); LC-MS M+H 580

NMR: CDCl₃ 0.75 (3H, d) 0.85 (3H, d) 1.1-2.1 (14H, m) 2.2-2.5 (7H, m) 2.55 (1H, t) 2.65 (3H, s) 2.7-2.9 (8H, m) 2.95 (1H, d) 3.05 (2H, m) 3.6 (1H, m) 3.7-3.9 (2H, m) 6.6-6.9 (3H, m) 7.2 (1H, m)

### EXAMPLE 3c

Using tert-butyl 4-{2-[ethyl(piperidin-4-yl)amino]-2-oxoethyl}piperazine-1-carboxylate was prepared tert-butyl 4-{2-[(1-{(3R)-3-(3,5-difluorophenyl)-3-[1-(methylsulfonyl)piperidin-4-yl]propyl}piperidin-4-yl)(methyl)amino]-2-oxoethyl}piperazine-1-carboxylate (Example 1c) as starting material was prepared N-(1{(3R)-3-(3,5-difluorophenyl)-3-[1-(methylsulfonyl)piperidin-4-yl]propyl}piperidin-4-yl)-N-methyl-2-piperazin-1-ylacetamide; LC-MS M+H 556

### EXAMPLE 4

This Example describes the preparation of N-(1-{(3R)-3-(3,5-difluorophenyl)-3-[1-(methylsulfonyl)piperidin-4-yl]propyl}piperidin-4-yl)-N-ethyl-2-[4-(methylsulfonyl)piperazin-1-yl]acetamide (Compound 5 Table I).

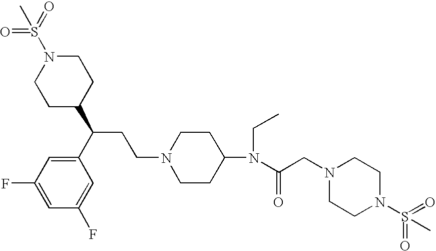

Methanesulphonyl chloride (55 μl) was added to a solution of N-(1-{(3R)-3-(3,5-difluorophenyl)-3-[1-(methylsulfonyl)piperidin-4-yl]propyl}piperidin-4-yl)-N-ethyl-2-piperazin-1-ylacetamide (200 mg) and triethylamine (143 μl) in dichloromethane (3.5 ml) at 0° C. under argon. The reaction mixture was allowed to warm to room temperature and stirring was continued for 3 hours. The reaction mixture was diluted with dichloromethane (15 ml) and washed with saturated ammonium chloride solution (2×10 ml) and brine (1×10 ml) and dried. The solvent was evaporated to give the title compound as a white foam, yield 185 mg, LC-MS M+H 648.

¹H NMR (CDCl₃): 1.10-2.98 (27H, m), 2.74 (3H, s), 2.78 (3H, s), 3.18-3.32 (8H, m), 3.62 & 4.34 (1H, m), 3.72 (1H, d), 3.84 (1H, d), 6.64 (3H, m).

Using this procedure and starting with N-(1-{(3R)-3-(3,5-difluorophenyl)-3-[1-(methylsulfonyl)piperidin-4-yl]propyl}piperidin-4-yl)-N-ethyl-2-piperidin-4-ylacetamide (Example 3) there is obtained N-(1-{(3R)-3-(3,5-difluorophenyl)-3-[1-(methylsulfonyl)piperidin-4-yl]propyl}piperidin-4-yl)-N-ethyl-2-[1-(methylsulfonyl)piperidin-4-yl]acetamide (Compound 6 Table I) LC-MS M+H 647, ¹H NMR (CDCl₃): 1.08-2.96 (32H, m), 2.72 (3H, s), 2.74 (3H, s), 3.26 (2H, m), 3.48 & 4.38 (1H, m), 3.72 (1H, d), 3.82 (3H, m), 6.64 (3H, m).

Using this procedure and starting with N-(1-{(3R)-3-(3,5-difluorophenyl)-3-[1-(1-ethylsulfonyl)piperidin-4-yl]propyl}piperidin-4-yl)-N-isobutyl-2-piperazin-1-ylacetamide (Example 3a) there is obtained N-(1-{(3R)-3-(3,5-difluorophenyl)-3-[1-(methylsulfonyl)piperidin-4-yl]propyl}piperidin-4-yl)-N-isobutyl-2-[4-(methylsulfonyl)piperazin-1-yl]acetamide (Compound 14 Table I) LC-MS M+H 676, ¹H NMR (CDCl₃): 0.8 (d, 3H) 0.9 (d, 3H) 1.1-2.2 (m, 16H) 2.4-2.7 (m, 7H) 2.75 (s, 3H) 2.8 (s, 3H) 2.85-3.1 (m, 4H) 3.2-3.4 (m, 6H) 3.6 (m, 1H) 3.7 (m, 1H) 3.8 (m, 1H) 6.6 (m, 3H).

Using this procedure and starting with N-(1-{(3R)-3-(3-fluorophenyl)-3-[1-(methylsulfonyl)piperidin-4-yl]propyl}piperidin-4-yl)-N-isobutyl-2-piperazin-1-ylacetamide (Example 3b) there is obtained N-(1-{(3R)-3-(3-fluorophenyl)-3-[1-(methylsulfonyl)piperidin-4-yl]propyl}piperidin-4-yl)-N-isobutyl-2-[4-(methylsulfonyl)piperazin-1-yl]acetamide (Compound 17 Table I) LC-MS M+H 658, ¹H NMR (CDCl₃): 0.85 (3H, d), 0.95 (3H, d), 1.2-2.2 (17H, m), 2.4 (1H, m), 2.5 (1H, m), 2.6-2.7 (5H, m), 2.75 (3H, s), 2.78 (3H, s), 2.8-2.95 (2H, m), 3.05 (2H, m), 3.2-3.35 (6H, m), 3.6, 4.05 (1H, m), 3.7 (1H, m), 3.85 (1H, m), 6.75-6.95 (3H, m), 7.2 (1H, m).

Using this procedure and starting with N-(cyclopropylmethyl)-N-(1-{(3R)-3-(3,5-difluorophenyl)-3-[1-(methylsulfonyl)piperidin-4-yl]propyl}piperidin-4-yl)-2-piperazin-1-ylacetamide (Method H) there is obtained N-(cyclopropylmethyl)-N-(1-{(3R)-3-(3,5-difluorophenyl)-3-[1-(methylsulfonyl)piperidin-4-yl]propyl}piperidin-4-yl)-2-[4-(methylsulfonyl)piperazin-1-yl]acetamide (Compound 22 Table I) LC-MS M+H 674, ¹H NMR (CDCl₃): 0.2 (d, 2H), 0.4 (d, 1H), 0.6 (d, 1H), 1.5-1.1 (m, 10H), 2.1-1.7 (m, 6H), 1.55 (br, 3H), 2.6-2.3 (m, 6H), 2.55 (s, 3H), 2.6 (s, 3H), 2.9-2.78 (m, 2H), 3.3-3.1 (m, 6H), 3.5 (m, 1H), 3.7 (d, 1H), 3.8 (d, 1H), 6.65 (m, 3H).

Using this procedure and starting with N-(1-{(3R)-3-(3,5-difluorophenyl)-3-[1-(methylsulfonyl)piperidin-4-yl]propyl}piperidin-4-yl)-N-methyl-2-piperazin-d-ylacetamide (Example 3c) there is obtained N-(1-{(3R)-3-(3,5-difluorophenyl)-3-[1-(methylsulfonyl)piperidin-4-yl]propyl}piperidin-4-yl)-N-methyl-2-[1-(methylsulfonyl)piperidin-4-yl]acetamide (Compound 24 Table I) LC-MS M+H 634, ¹H NMR (CDCl₃): 1.2-2.2 (16H, m) 2.4 (1H, m) 2.5 (1H, m) 2.6-2.7 (5H, m) 2.75 (3H, s) 2.8 (3H, s) 2.85 (1H, m) 2.9-2.95 (3H, m) 3.2-3.3 (6H, m) 3.6, 4.4 (1H, m) 3.75 (1H, m) 3.85 (1H, m) 6.7 (3H, m).

Using this procedure and starting with N-(1-{(3R)-3-(3,5-difluorophenyl)-3-[1-(methylsulfonyl)piperidin-4-yl]propyl}piperidin-4-yl)-N-isobutyl-N′-piperidin-4-ylurea (Method K) there is obtained N-(1-{(3R)-3-(3,5-difluorophenyl)-3-[1-(methylsulfonyl)piperidin-4-yl]propyl}piperidin-4-yl)-N-isobutyl-N-[1-(methylsulfonyl)piperidin-4-yl]urea (Compound 1 Table VI). LC-MS M+H 676, ¹H NMR (CDCl₃): 0.9 (6H, d) 1.2-2.2 (24H, m) 2.4 (1H, m) 2.5 (1H, m) 2.6 (1H, m) 2.7 (3H, s) 2.75-2.8 (4H, m) 2.9 (2H, m) 3.7-4 (4H, m) 4.25 (1H, d) 6.7 (3H, m).

Using this procedure and starting with N-(1-{(3R)-3-(3,5-difluorophenyl)-3-[1-(methylsulfonyl)piperidin-4-yl]propyl}piperidin-4-yl)-N-methyl-N′-piperidin-4-ylurea (Method K) there is obtained N-(1-{(3R)-3-(3,5-difluorophenyl)-3-[1-(methylsulfonyl)piperidin-4-yl]propyl}piperidin-4-yl)-N-methyl-N-[1-(methylsulfonyl)piperidin-4-yl]urea (Compound 4 Table VI). LC-MS M+H 634, ¹H NMR (CDCl₃): 1.3-1.7 (7H, m) 1.9-2.3 (8H, m) 2.4 (1H, m) 2.5 (1H, m) 2.6 (1H, m) 2.7 (7H, m) 2.8 (5H, m) 2.85-2.9 (3H, m) 3.7-3.9 (6H, m) 4.1 (1H, m) 4.2 (1H, d) 6.65 (3H, m).

### EXAMPLE 5

This Example describes the preparation of N-(1-{(3R)-3-(3,5-difluorophenyl)-3-[1-(methylsulfonyl)piperidin-4-yl]propyl}piperidin-4-yl)-N-ethyl-N′-{[1-(methylsulfonyl)piperidin-4-yl]methyl}urea (Compound 1 Table II).

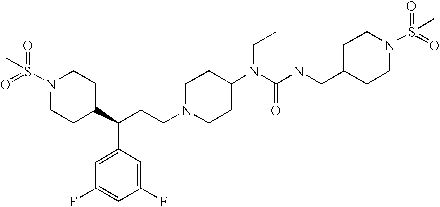

PS-triacetoxyborohydride (694 mg) was added to a solution of N-ethyl-N′-{[1-(methylsulfonyl)piperidin-4-yl]methyl}-N-piperidin-4-ylurea (173 mg) in dichloromethane (10 ml) and stirred for 18 hours. The reaction mixture was filtered and the filtrate was washed with saturated sodium bicarbonate solution (1×15 ml) and brine (1×15 ml) and dried. The residue obtained on removal of the solvent was purified on a 20 g silica Bond Elut eluted with a solvent gradient of ethyl acetate-30% methanol/ethyl acetate and the material obtained was passed through a 20 g SCX2 column eluting with methanol initially, then with 10% 7M ammonia in methanol. Evaporation of the methanolic ammonia washings gave the title compound as a white foam, yield 120 mg, LC-MS M+H 662.

¹H NMR (CDCl₃): 1.2 (t, 3H) 1.2-2.1 (m, 19H) 2.4-2.7 (m, 5H) 2.8 (m, 6H) 2.9 (m, 2H) 3.2 (m, 4H) 3.7-3.9 (m, 4H) 4.1 (m, 1H) 4.5 (m, 1H) 6.6 (m, 3H).

### EXAMPLE 6

This Example describes the preparation of N-allyl-N-(1-{(3R)-3-(3,5-difluorophenyl)-3-[1-(methylsulfonyl)piperidin-4-yl]propyl}piperidin-4-yl)-2-[1-(methylsulfonyl)piperidin-4-yl]acetamide (Compound 7 Table I).

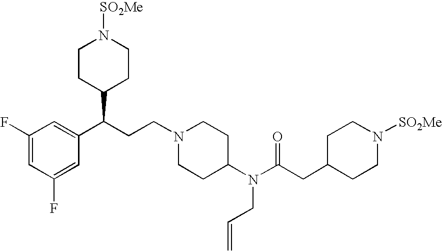

To a solution of (R) 3-(N-methanesulphonylpiperidin-4-yl)-3-(3,5-difluorophenyl)propionaldehyde (300 mg) in CH₂Cl₂ (40 ml) was added N-allyl-2-[1-(methylsulfonyl)piperidin-4-yl]-N-piperidin-4-ylacetamide hydrochloride (412 mg), triethylamine (0.15 ml) and sodium triacetoxyborohydride (471 mg) and the mixture stirred at room temperature for 18 h. The reaction mixture was washed with aqueous sodium bicarbonate (30 ml) then brine (30 ml) and dried (MgSO₄). Preparative HPLC (acetonitrile/water) gave the product as a white solid (340 mg), MH⁺ (659).

NMR (DMSO): 1.0-1.3 (m, 2H), 1.35 (d, 1H), 1.5-2.0 (m, 6H), 2.0-2.5 (m, 5H), 2.5-2.7 (m, 2H), 2.81 (s, 3H), 2.83 (s, 3H), 2.8-3.1 (m, 1H), 3.3-3.7 (m, 5H), 3.7-4.0 (m, 9H), 4.5 (t, 1H), 5.0-5.2 (m, 2H), 5.8-5.9 (m, 1H), 7.0 (m, 2H), 7.1 (t, 1H).

### EXAMPLE 7

This Example describes the preparation of N-(1-{(3R)-3-(3,5-difluorophenyl)-3-[1-(methylsulfonyl)piperidin-4-yl]propyl}piperidin-4-yl)-2-[1-(methylsulfonyl)piperidin-4-yl]-N-propylacetamide (Compound 8 Table I).

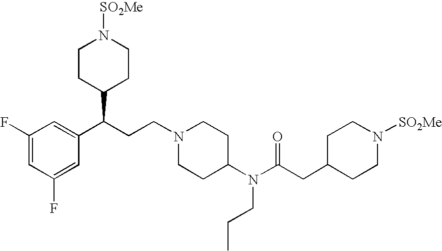

A suspension of N-allyl-N-(1-{(3R)-3-(3,5-difluorophenyl)-3-[1-(methylsulfonyl)piperidin-4-yl]propyl}piperidin-4-yl)-2-[1-(methylsulfonyl)piperidin-4-yl]acetamide (50 mg) and palladium on carbon (10%, 20 mg) in ethanol (20 ml) was stirred under a hydrogen atmosphere at room temperature for 18 h. The reaction mixture was filtered through a plug of Celite® and the filtrate concentrated under reduced pressure to provide the product as a white solid (50 mg). MH⁺ (661).

NMR (DMSO): 0.8 (m, 2H), 0.9 (t, 3H), 1.0-1.4 (m, 4H), 1.4-1.8 (m, 4H), 1.8-2.2 (m, 6H), 2.2-2.4 (m, 6H), 2.6-2.8 (m, 6H), 2.85 (s, 3H), 2.9 (s, 3H), 2.9-3.1 (m, 4H), 3.4-3.6 (m, 6H), 6.9-7.0 (m, 2H), 7.1 (t, 1H).

### EXAMPLE 8

This Example describes the preparation of N-(1-{(3R)-3-(3,5-difluorophenyl)-3-[4-(methylsulphonyl)phenyl]propyl}piperidin-4-yl)-N-ethyl-2-[1-(methylsulphonyl)piperidin-4-yl]acetamide (Compound 1 Table III).

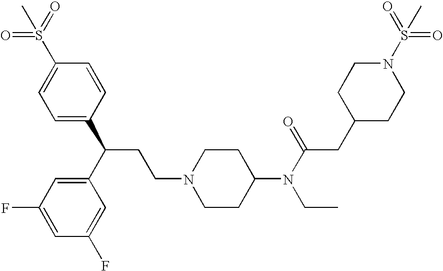

Diisopropylethylamine (130 μl) was added to a suspension of [1-(methylsulphonyl)piperidin-4-yl]acetic acid [CAS 423722-27-4] (111 mg) and HATU (228 mg) in dichloromethane (3 ml) and the mixture was stirred for 15 minutes. A solution of 1-{(3R)-3-(3,5-difluorophenyl)-3-[4-(methylsulphonyl)phenyl]propyl}-N-ethylpiperidin-4-amine (218 mg) in dichloromethane (2 ml) was added and the mixture was stirred for 16 hours. The reaction mixture was diluted with dichloromethane (15 ml) and washed with water (2×20 ml), saturated aqueous sodium bicarbonate solution (2×20 ml), brine (10 ml) and dried. The solvent was evaporated and the residue purified on a silica column eluting with a solvent gradient of ethyl acetate-20% methanol/ethyl acetate, yield 184 mg, M+H 640.

In an analogous manner but using tetrahydro-2H-pyran-4-ylacetic acid [CAS 85064-61-5] as starting material there is obtained N-(1-{(3R)-3-(3,5-difluorophenyl)-3-[4-(methylsulphonyl)phenyl]propyl}piperidin-4-yl)-N-ethyl-2-(tetrahydro-2H-pyran-4-yl)acetamide, M+H 563, (Compound 2 Table III).

In an analogous manner but using tetrahydro-2H-pyran-4-ylacetic acid [CAS 85064-61-5] and 1-{(3R)-3-(3,5-difluorophenyl)-3-[1-(methylsulfonyl)piperidin-4-yl]propyl}-N-isobutylpiperidin-4-amine (Method H) as starting material there was obtained N-(1-{(3R)-3-(3,5-difluorophenyl)-3-[1-(methylsulfonyl)piperidin-4-yl]propyl}piperidin-4-yl)-N-isobutyl-2-(tetrahydro-2H-pyran-4-yl)acetamide (Compound 1 Table V). LC-MS M+H 598, ¹H NMR (CDCl₃): 0.75 (d, 3H) 0.8 (d, 3H) 1.1-2.2 (m, 26H) 2.3 (m, 1H) 2.4 (t, 1H) 2.6 (t, 1H) 2.7 (s, 3H) 2.75-2.9 (m, 2H) 3.4-3.5 (m, 2H) 3.65 (m, 1H) 3.8 (m, 1H) 3.85 (m, 2H) 6.6 (m, 3H).

In an analogous manner but using tetrahydrofuran-2-ylacetic acid and 1-{(3R)-3-(3,5-difluorophenyl)-3-[1-(methylsulfonyl)piperidin-4-yl]propyl}-N-isobutylpiperidin-4-amine (Method H) as starting material there was obtained N-(1-{(3R)-3-(3,5-difluorophenyl)-3-[1-(methylsulfonyl)piperidin-4-yl]propyl} piperidin-4-yl)-N-isobutyl-2-(tetrahydrofuran-2-yl)acetamide (Compound 2 Table V). LC-MS M+H 584, ¹H NMR (CDCl₃): 0.75 (3H, d) 0.85 (3H, d) 1.1-2.15 (22H, m) 2.35 (2H, m) 2.45 (1H, m) 2.55 (1H, m) 2.65 (4H, m) 2.7-2.8 (2H, m) 3.5, 4.1 (1H, m) 3.65 (2H, m) 3.8 (2H, m) 4.2 (1H, m) 6.6 (3H, m).

In an analogous manner but using (1,1-dioxidothiomorpholin-4-yl)acetic acid and 1-{(3R)-3-(3,5-difluorophenyl)-3-[1-(methylsulfonyl)piperidin-4-yl]propyl}-N-isobutylpiperidin-4-amine (Method H) as starting material there was obtained N-(1-{(3R)-3-(3,5-difluorophenyl)-3-[1-(methylsulfonyl)piperidin-4-yl]propyl}piperidin-4-yl)-2-(1,1-dioxidothiomorpholin-4-yl)-N-isobutylacetamide (Compound 3 Table V). LC-MS M+H 647, ¹H NMR (CDCl₃): 0.9 (dt, 6H), 1.4-2.0 (m, 23H), 2.4-2.6 (m, 4H), 2.7 (s, H), 3.1 (br, 5H), 3.35 (s, 2H), 3.7 (d, 1H), 3.85 (d, 1H), 6.65 (m, 3H).

### EXAMPLE 9

This Example describes the preparation of 2-(4-acetylpiperazin-1-yl)-N-(1-{(3R)-3-(3,5-difluorophenyl)-3-[1-(methylsulphonyl)piperidin-4-yl]propyl}piperidin-4-yl)-N-ethylacetamide (Compound 9 Table I).

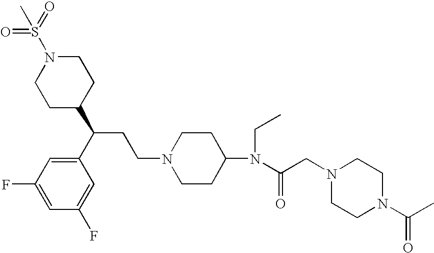

Acetic anhydride (66 μl) was added to a stirred solution of N-(1-{(3R)-3-(3,5-difluorophenyl)-3-[1-(methylsulphonyl)piperidin-4-yl]propyl}piperidin-4-yl)-N-ethyl-2-piperazin-1-ylacetamide (200 mg) [Example 3, part 2] and triethylamine (143 μl) in dichloromethane (3.5 ml) at 0° C. under an argon atmosphere. The mixture was allowed to warm to room temperature and was stirred for 16 hours. The reaction mixture was diluted with dichloromethane (10 ml) and washed with ammonium chloride solution (2×10 ml) and brine (10 ml) and dried. The residue obtained on evaporation of the solvent was purified by chromatography on a 12 g silica cartridge eluting with a solvent gradient of ethyl acetate-40% methanol/ethyl acetate, yield 105 mg, M+H 612.

In an analogous manner but using N-(1-{(3R)-3-(3,5-difluorophenyl)-3-[1-(methylsulphonyl)piperidin-4-yl]propyl}piperidin-4-yl)-N-ethyl-2-piperidin-4-ylacetamide as starting material (Example 3), there is obtained 2-(1-acetylpiperidin-4-yl)-N-(1-{(3R)-3-(3,5-difluorophenyl)-3-[1-(methylsulphonyl)piperidin-4-yl]propyl}piperidin-4-yl)-N-ethylacetamide (Compound 10 Table I).

In an analogous manner but using N-(1-{(3R)-3-(3,5-difluorophenyl)-3-[1-(methylsulfonyl)piperidin-4-yl]propyl}piperidin-4-yl)-N-isobutyl-2-piperazin-1-ylacetamide (Compound 13, Table I)) as starting material there is obtained 2-(4-acetylpiperazin-1-yl)-N-(1-{(3R)-3-(3,5-difluorophenyl)-3-[1-(methylsulfonyl)piperidin-4-yl]propyl}piperidin-4-yl)-N-isobutylacetamide (Compound 15 table I) LC-MS M+H 640, ¹H NMR (CDCl₃): 0.8 (d, 3H) 0.9 (d, 3H) 1.1-2.2 (m, 24H) 2.4-2.65 (m 7H) 2.7 (m, 3H) 2.8-3.2 (m, 4H) 3.45 (m, 1H) 3.6 (m, 1H) 3.7 (m, 1H) 3.8 (m, 1H).

In an analogous manner but using N-(1-{(3R)-3-(3-fluorophenyl)-3-[1-(methylsulfonyl)piperidin-4-yl]propyl}piperidin-4-yl)-N-isobutyl-2-piperazin-1-ylacetamide (Compound 16 Table I) as starting material there is obtained 2-(4-acetylpiperazin-1-yl)-N-(1-{(3R)-3-(3-fluorophenyl)-3-[1-(methylsulfonyl)piperidin-4-yl]propyl}piperidin-4-yl)-N-isobutylacetamide (Compound 19 table I) LC-MS M+H 622, ¹H NMR (CDCl₃): 0.85 (3H, d) 0.95 (3H, d) 1.2-2.0 (15H, m) 2.05 (3H, s) 2.1 (1H, m) 2.35 (1H, m) 2.5-2.65 (6H, m) 2.75 (3H, s) 2.8-2.95 (2H, m) 3.1 (2H, m) 3.2 (2H, m) 3.45 (2H, m) 3.5, 4.05 (1H, m) 3.6-3.75 (3H, m) 3.85 (1H, m) 6.8-6.95 (3H, m) 7.25 (1H, m).

In an analogous manner but using N-(cyclopropylmethyl)-N-(1-{(3R)-3-(3,5-difluorophenyl)-3-[1-(methylsulfonyl)piperidin-4-yl]propyl}piperidin-4-yl)-2-piperazin-1-ylacetamide (Method H) as starting material there is obtained 2-(4-acetylpiperazin-1-yl)-N-(cyclopropylmethyl)-N-(1-{(3R)-3-(3,5-difluorophenyl)-3-[1-(methylsulfonyl)piperidin-4-yl]propyl}piperidin-4-yl)acetamide (Compound 23 Table I) LC-MS M+H 638, ¹H NMR (CDCl₃): 0.3 (d, 2H), 0.5 (d, 1H), 0.65 (d, 1H), 1.2-1.55 (m, 8H), 1.65 (br, 3H), 1.75-2.05 (m, 4H), 2.1 (s, 3H), 2.4-2.7 (m, 8H), 2.75 (s, 3H), 2.85-2.95 (m, 2H), 3.0-3.3 (m, 5H), 3.45-3.6 (m, 4H), 3.75 (d, 1H), 3.85 (d, 1H), 6.65 (m, 3H).

In an analogous manner but using N-(1-{(3R)-3-(3,5-difluorophenyl)-3-[1-(methylsulfonyl)piperidin-4-yl]propyl}piperidin-4-yl)-N-methyl-2-piperazin-1-ylacetamide (Example 3c) as starting material there is obtained 2-(4-acetylpiperazin-1-yl)-N-(1-{(3R)-3-(3,5-difluorophenyl)-3-[1-(methylsulfonyl)piperidin-4-yl]propyl}piperidin-4-yl)-N-methylacetamide (Compound 26 Table I) LC-MS M+H 598, ¹H NMR (CDCl₃): 1.2-2.05 (15H, m) 2.1 (3H, s) 2.4 (1H, m) 2.45-2.65 (6H, m) 2.7 (3H, s) 2.8-2.95 ((5H, m) 3.2 (2H, d) 3.4-3.5 ((2H, m) 3.6-3.7 (2H, m) 3.75 (1H, m) 3.85 (1H, m) 4.4 (1H, m) 6.7 (3H, m).

In an analogous manner but using N-(1-{(3R)-3-(3,5-difluorophenyl)-3-[1-(methylsulfonyl)piperidin-4-yl]propyl}piperidin-4-yl)-N-isobutyl-1-piperidin-4-ylurea (Method K) as starting material there is obtained N-(1-acetylpiperidin-4-yl)-N-(1-{(3R)-3-(3,5-difluorophenyl)-3-[1-(methylsulfonyl)piperidin-4-yl]propyl}piperidin-4-yl)-N-isobutylurea (Compound 3 Table VI)) LC-MS M+H 640, ¹H NMR (CDCl₃): 0.95 (6H, d) 1.2-2.1 (21H, m) 2.15 (3H, s) 2.35 (1H, m) 2.5 (1H, m 2.6 (1H, m) 2.7 (4H, m) 2.8-2.9 (4H, m) 3.15 (1H, m) 3.75 (2H, m) 3.8-4 (3H, m) 4.2 (1H, d) 4.5 (1H, m) 6.65 (3H, m).

### EXAMPLE 10

This Example describes the preparation of N-(1-{(3R)-3-(3,5-difluorophenyl)-3-[4-(methylsulphonyl)phenyl]propyl}piperidin-4-yl)-N-ethyl-2-morpholin-4-ylacetamide (Compound 3 Table III).

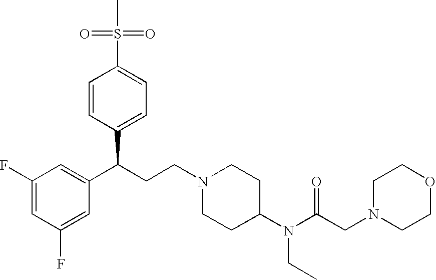

### Step 1: Preparation of 2-bromo-N-(1-{(3R)-3-(3,5-difluorophenyl)-3-[4-(methylsulphonyl)phenyl]propyl}piperidin-4-yl)-N-ethylacetamide

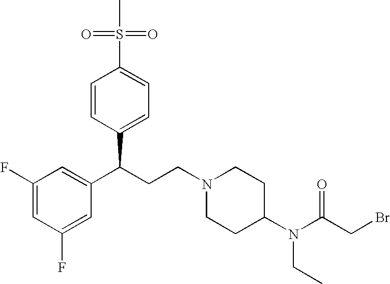

A solution of 1-{(3R)-3-(3,5-difluorophenyl)-3-[4-(methylsulphonyl)phenyl]propyl}-N-ethylpiperidin-4-amine (Method F) (314 mg) and triethylamine (200 μl) in dichloromethane (3.5 ml) was added to a stirred solution of bromoacetyl chloride (72 μl) in dichloromethane (3.5 ml) under argon at 0° C. The mixture was allowed to warm to room temperature and stirring was continued for 2 hours. The reaction mixture was diluted with dichloromethane (10 ml) washed with ammonium chloride solution (2×15 ml), brine (15 ml) and dried. The brown foam (339 mg) [M+H 558] obtained on evaporation of the solvent was used directly in Step 2.

### Step 2: Preparation of the Title Compound

A mixture of 2-bromo-N-(1-{(3R)-3-(3,5-difluorophenyl)-3-[4-(methylsulphonyl)phenyl]propyl}piperidin-4-yl)-N-ethylacetamide (170 mg), morpholine (53 μl) and potassium carbonate (126 mg) in dioxane (3.1 ml) was stirred at room temperature for 30 minutes then warmed to 50° C. for 1 hour. The solvent was evaporated and the residue was dissolved in dichloromethane (15 ml), washed with water (2×15 ml), brine (15 ml) and dried. The residue obtained on evaporation of the solvent was purified on a 12 g silica cartridge eluting with a solvent gradient of ethyl acetate-30% methanol/ethyl acetate (yield 58 mg, M+H 564).

### EXAMPLE 11

This Example describes the preparation of N-[1-((3R)-3-(3,5-difluorophenyl)-3-{1-[(trifluoromethyl)sulfonyl]piperidin-4-yl}propyl)piperidin-4-yl]-N-ethyl-2-[1-(methylsulfonyl)piperidin-4-yl]acetamide (Compound 11 Table I)

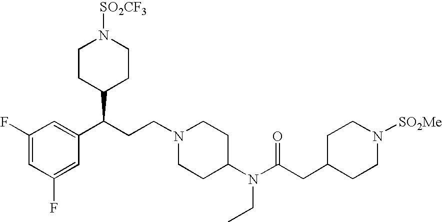

### Step 1: Preparation of tert-butyl 4-{2-[[1-((3R)-3-(3,5-difluorophenyl)-3-{1-[(trifluoromethyl)sulfonyl]piperidin-4-yl}propyl)piperidin-4-yl](ethyl)amino]-2-oxoethyl}piperidine-1-carboxylate

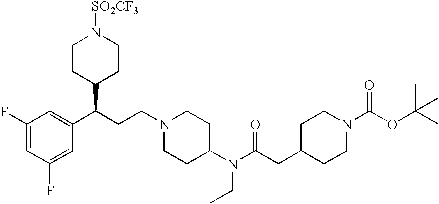

To a solution of (3R)-3-(3,5-difluorophenyl)-3-{1-[(trifluoromethyl)sulfonyl]piperidin-4-yl}propanal (271 mg), (Method A) and tert-butyl 4-{2-[ethyl(piperidin-4-yl)amino]-2-oxoethyl}piperidine-1-carboxylate (Method C) in dichloromethane (7 ml) was added glacial acetic acid (50 μL) and sodium triacetoxyborohydride (178 mg) and the resulting mixture was stirred for 18 hours. The mixture was quenched with sodium bicarbonate solution and extracted with dichloromethane. The organics were dried and evaporated to a give a solid which was purified by silica chromatography eluting with a gradient of ethyl acetate/isohexane (0 to 20%) to give the subtitled compound as a white solid (yield 270 mg) M+H 723.

### Step 2: Preparation of the Title Compound

To a solution of tert-butyl 4-{2-[[1-((3R)-3-(3,5-difluorophenyl)-3-{1-[(trifluoromethyl)sulfonyl]piperidin-4-yl}propyl)piperidin-4-yl](ethyl)amino]-2-oxoethyl}piperidine-1-carboxylate (270 mg) in methanol (1 ml) was added 4N HCl in dioxane (4 ml) and the resulting mixture was stirred for 1 hour. The mixture was concentrated and partitioned between dichloromethane and 2M NaOH and the aqueous was extracted with further dichloromethane (3×). The organics were dried and evaporated to dryness. The residue was dissolved in dichloromethane (4 ml), cooled to 5° C. under an argon atmosphere. Triethylamine (104 μl) and methanesulphonyl chloride (44 μl) was added and the mixture was allowed to warm to room temperature and to stir for 18 hours. The mixture was diluted with dichloromethane and washed with saturated ammonium chloride (2×). The organics were dried and evaporated to a gum which was purified by silica chromatography eluting with a gradient of methanol/dichloromethane (0:100 to 20:80) to give the titled compound as a white solid (121 mg). LC-MS M+H 701 ¹H NMR (CDCl₃): 1.06-2.29 (m, 26H), 2.42 (m, 1H), 2.67 (t, 2H), 2.76 (s, 3H), 2.78 (m, 1H), 2.89 (t, 1H), 3.00 (t, 1H), 3.28 (m, 2H), 3.48 and 4.37 (m, 1H), 3.78 (m, 2H), 3.87 (m, 1H), 4.00 (m, 1H), 6.66 (m, 3H).

### EXAMPLE 12

This Example describes the preparation of methyl 4-{2-[(1-{(3R)-3-(3-fluorophenyl)-3-[1-(methylsulfonyl)piperidin-4-yl]propyl}piperidin-4-yl)(isobutyl)amino]-2-oxoethyl}piperazine-1-carboxylate (Compound 18 Table I)

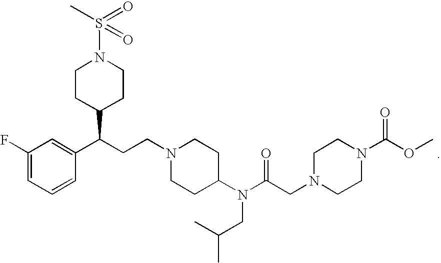

To a solution of N-(1-{(3R)-3-(3-fluorophenyl)-3-[1-(methylsulfonyl)piperidin-4-yl]propyl}piperidin-4-yl)-N-isobutyl-2-piperazin-1-ylacetamide (Compound 16 Table I) (329 mg) and triethylamine (104 uL) in dichloromethane (25 ml) was added methyl chloroformate (53 uL). The reaction mixture was stirred at room temperature for 24 hours, washed with 2N NaOH (2×25 ml), dried over MgSO₄ and evaporated. The residue was purified by silica chromatography eluting with a gradient of ethyl acetate to 35% methanol/ethyl acetate to give the title compound as a solid. Yield 159 mg. LC-MS M+H 638 ¹H NMR (CDCl₃): 0.85 (3H, d) 0.95 (3H, d) 1.2-2.2 (18H, m) 2.4 (1H, m) 2.5 (4H, m) 2.6 (1H, m) 2.7 (3H, s) 2.8-2.95 (2H, m) 3.05 (2H, m) 3.2 (2H, m) 3.45 (3H, m) 3.5, 4.05 (1H, m) 3.7 (4H, m) 3.85 (1H, m) 6.8-6.95 (3H, m) 7.25 (1H, m).

In an analogous manner but using N-(1-{(3R)-3-(3,5-difluorophenyl)-3-[1-(methylsulfonyl)piperidin-4-yl]propyl}piperidin-4-yl)-N-methyl-2-piperazin-1-ylacetamide (Example 3c) as starting material there was obtained methyl 4-{2-[(1-{(3R)-3-(3,5-difluorophenyl)-3-[1-(methylsulfonyl)piperidin-4-yl]propyl}piperidin-4-yl)(methyl)amino]-2-oxoethyl}piperazine-1-carboxylate (Compound 25 Table I) LC-MS M+H 614, ¹H NMR (CDCl₃): 1.15-2.1 (16H, m) 2.35-2.5 (5H, m) 2.6 (1H, m) 2.75 (3H, s) 2.8-2.95 (5H, m) 3.15 (2H, d) 3.45-3.55 (4H, m) 3.7 (4H, m) 3.75, 4.4 (1H, m) 3.85 (1H, m) 6.7 (3H, m).

In an analogous manner but using N-(1-{(3R)-3-(3,5-difluorophenyl)-3-[1-(methylsulfonyl)piperidin-4-yl]propyl}piperidin-4-yl)-N-isobutyl-2-piperazin-1-ylacetamide (Compound 13 Table I) as starting material there was obtained methyl 4-{2-[(1-{(3R)-3-(3,5-difluorophenyl)-3-[1-(methylsulfonyl)piperidin-4-yl]propyl}piperidin-4-yl)(isobutyl)amino]-2-oxoethyl}piperazine-1-carboxylate (Compound 27 Table I) LC-MS M+H 656, ¹H NMR (CDCl₃): 0.35 (m, 3H), 0.9 (m, 3H), 1.2-2.1 (m, 17H), 2.35-2.55 (m, 6H), 2.65 (m, 1H), 2.8 (s, 3H), 2.9 (m, 2H), 3.1 (m, 2H), 3.2 (d, 2H), 3.5 (m, 4H), 3.7 (s, 3H), 3.75 (d, 1H), 3.85 (d, 1H), 6.65 (m, 3H).

In an analogous manner but using N-(cyclopropylmethyl)-N-(1-{(3R)-3-(3,5-difluorophenyl)-3-[1-(methylsulfonyl)piperidin-4-yl]propyl}piperidin-4-yl)-2-piperazin-1-ylacetamide (Method H) as starting material there was obtained methyl 4-{2-[(cyclopropylmethyl)(1-{(3R)-3-(3,5-difluorophenyl)-3-[1-(methylsulfonyl)piperidin-4-yl]propyl}piperidin-4-yl)amino]-2-oxoethyl}piperazine-1-carboxylate (Compound 28 Table I) LC-MS M+H 654, ¹H NMR (CDCl₃): 0.25 (m, 2H), 0.45 (d, 1H), 0.6 (d, 1H), 1.0-2.1 (m, 12H), 2.35-2.65 (m, 9H), 2.7 (s, 3H), 2.8 (s, 1H), 2.9 (m, 2H), 3.1-3.25 (m, 5H), 3.45 (m, 5) 3.7 (s, 3H), 3.75 (d, 1H), 3.85 (d, 1H), 6.65 (m, 3H).

In an analogous manner but using N-(1-{(3R)-3-(3,5-difluorophenyl)-3-[1-(methylsulfonyl)piperidin-4-yl]propyl}piperidin-4-yl)-N-isobutyl-N′-piperidin-4-ylurea (Method K) as starting material there was obtained methyl 4-({[(1-{(3R)-3-(3,5-difluorophenyl)-3-[1-(methylsulfonyl)piperidin-4-yl]propyl}piperidin-4-yl)(isobutyl)amino]carbonyl}amino)piperidine-1-carboxylate (Compound 2 Table VI). LC-MS M+H 656, ¹H NMR (CDCl₃): 0.9 (6H, d) 1.2-2.2 (19H, m) 2.35 (1H, m) 2.5 (1H, m) 2.6 (1H, m) 2.75 (3H, s) 2.8-2.95 (6H, m) 3.65 (3H, s) 3.7 (1H, m) 3.85 (2H, m) 3.9-4.15 (3H, m) 4.2 (1H, d) 6.7 (3H, m).

In an analogous manner but using N-(1-{(3R)-3-(3,5-difluorophenyl)-3-[1-(methylsulfonyl)piperidin-4-yl]propyl}piperidin-4-yl)-N-methyl-N′-piperidin-4-ylurea (Method K) as starting material there was obtained methyl 4-({[(1-{(3R)-3-(3,5-difluorophenyl)-3-[1-(methylsulfonyl)piperidin-4-yl]propyl}piperidin-4-yl)(methyl)amino]carbonyl}amino)piperidine-1-carboxylate (Compound 5 Table VI). LC-MS M+H 656, ¹H NMR (CDCl₃):

### EXAMPLE 13

This Example describes the preparation of N-(1-{(3R)-3-(3,5-difluorophenyl)-3-[1-(methylsulfonyl)piperidin-4-yl]propyl}piperidin-4-yl)-N-isobutyl-2-[4-(2-methoxyethyl)piperazin-1-yl]acetamide (Compound 21 Table I)

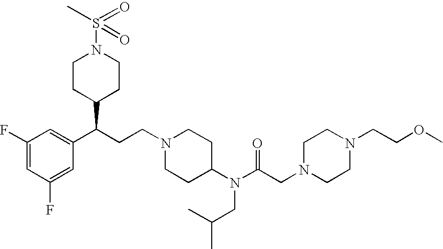

To a solution of N-(1-{(3R)-3-(3,5-difluorophenyl)-3-[1-(methylsulfonyl)piperidin-4-yl]propyl}piperidin-4-yl)-N-isobutyl-2-piperazin-1-ylacetamide (Compound 13 table I) (105 mg) and triethylamine (0.033 ml) in dichloromethane (10 ml) was added. 2-chloroethyl methyl ether (0.018 ml). The reaction mixture was stirred at room temperature for 18 hours, washed with 2N NaOH (2×10 ml), dried over MgSO4, filtered and evaporated to dryness. The residue was dissolved in MeOH (10 ml) and poured onto a SCX2 cartridge and eluted with MeOH (6×20 ml), then 1M NH3/methanol (6×50 ml). The combined ammonia fractions were evaporated to yield an oil which was transferred to a vial in dichloromethane/methanol solution, evaporated to dryness using a Genevac to yield an oil and high vacuumed dried to give the titled compound as a foam. Yield 44 mg LC-MS M+H 656, ¹H NMR (CDCl₃): 0.85 (d, 3H) 0.9 (d, 3H), 1.2-1.5 (m, 4H), 1.6-2.1 (m, 22H), 2.35-2.65 (m, 7H), 2.7 (s, 3H), 2.8-3.2 (m, 8H), 3.75 (d, 1H), 3.85 (d, 1H), 6.65 (m, 3H).

### EXAMPLE 14

This Example describes the preparation of N-(1-{(3R)-3-(3,5-difluorophenyl)-3-[1-(methylsulfonyl)piperidin-4-yl]propyl}piperidin-4-yl)-N-methyl-2-[1-(methylsulfonyl)piperidin-4-yl]acetamide (Compound 30 table I)

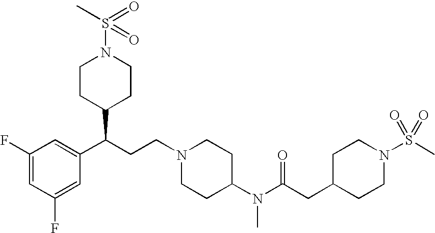

[1-(methylsulfonyl)piperidin-4-yl]acetic acid (117 mg) and HATU (202 mg) were dissolved in DMF (10 ml) and triethylamine (149 uL) was added. The reaction mixture was stirred at room temperature for 10 minutes. 1-{(3R)-3-(3,5-difluorophenyl)-3-[1-(methylsulfonyl)piperidin-4-yl]propyl}-N-methylpiperidin-4-amine (Method I) (250 mg) was added and the reaction stirred at room temperature for 18 hours. The solvent was removed by evaporation. The residue was dissolved in dichloromethane (25 ml) and washed with 2N NaOH (2×25 ml), dried over MgSO₄ and evaporated. The residue was purified by chromatography eluting with ethyl acetate to 30% methanol/ethyl acetate. Yield 105 mg LC-MS M+H 633, ¹H NMR (CDCl₃): 1.2-2.15 (21H, m) 2.25 (2H, d) 2.4 (1H, s) 2.5 (1H, s) 2.6-2.7 (3H, m) 2.75 (3H, s) 2.78 (3H, s) 2.8-2.95 (5H, m) 3.5, 4.45 (1H, m) 3.7-3.9 (4H, m) 6.65 (3H, m).

In an analogous manner but using 1-{(3R)-3-(3,5-difluorophenyl)-3-[1-(methylsulfonyl)piperidin-4-yl]propyl}-N-isobutylpiperidin-4-amine (Method I) there was obtained N-(1-{(3R)-3-(3,5-difluorophenyl)-3-[1-(methylsulfonyl)piperidin-4-yl]propyl}piperidin-4-yl)-N-isobutyl-2-[1-(methylsulfonyl)piperidin-4-yl]acetamide (Compound 29 Table I) LC-MS M+H 675, ¹H NMR (CDCl₃): 0.85 (3H, d) 0.9 (3H, d) 1.2-2.15 (21H, m) 2.25 (2H, d) 2.4 (1H, m) 2.5 (1H, m) 2.65 (3H, m) 2.75 (3H, s) 2.78 (3H, s) 2.8-2.9 (2H, m) 3.05 (1H, m) 3.1 (1H, m) 3.45, 4.1 (1H, m) 3.7-3.9 (4H, m) 6.65 (3H, m).

### EXAMPLE 15

This Example describes the preparation of N-(1-{(3R)-3-(3,5-difluorophenyl)-3-[1-(methylsulfonyl)piperidin-4-yl]propyl}piperidin-4-yl)-N-isobutyl-2-[1-(methylsulfonyl)pyrrolidin-3-yl]acetamide (Compound 4 Table V)

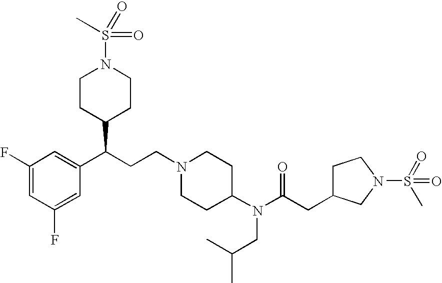

[1-(methylsulfonyl)pyrrolidin-3-yl]acetic acid (Method J) (93 mg) was dissolved in dichloromethane (20 ml) and carbonyldiimidazole (73 mg) was added. The reaction mixture was stirred at room temperature for 2 hours. 1-{(3R)-3-(3,5-difluorophenyl)-3-[1-(methylsulfonyl)piperidin-4-yl]propyl}-N-isobutylpiperidin-4-amine (Method I) (212 mg) was then added and the mixture stirred at room temperature for 24 hours. The solvent was evaporated and the residue dissolved in dichloromethane (25 ml) and washed with 2N NaOH (2×20 ml), dried over MgSO4 and evaporated. The residue was purified by silica chromatography, eluting with a gradient of ethyl acetate to 30% methanol/ethyl acetate to give the titled compound as a white foam. Yield 16 mg. LC-MS M+H 661.

In an analogous manner but using 1-{(3R)-3-(3,5-difluorophenyl)-3-[1-(methylsulfonyl)piperidin-4-yl]propyl}-N-ethylpiperidin-4-amine there was obtained N-(1-{(3R)-3-(3,5-difluorophenyl)-3-[1-(methylsulfonyl)piperidin-4-yl]propyl}piperidin-4-yl)-N-ethyl-2-[1-(methylsulfonyl)pyrrolidin-3-yl]acetamide (Compound 5 Table V). LC-MS M+H 633, ¹H NMR (CDCl₃): 1-2.2 (m, 23H) 2.3-2.6 (m, 5H) 2.7 (s, 3H) 2.75 (s, 3H) 2.8-2.9 (m, 2H) 3.2 (m, 3H) 3.35-3.5 (m, 2H) 3.65 (m, 1H) 3.8 (m, 1H) 6.6 (m, 3H).

### EXAMPLE 16

This Example describes the preparation N-(1-{(3R)-3-(3,5-difluorophenyl)-3-[1-(methylsulfonyl)piperidin-4-yl]propyl}piperidin-4-yl)-N-ethyl-2-[(2S)-(methylsulfonyl)pyrrolidin-2-yl]acetamide

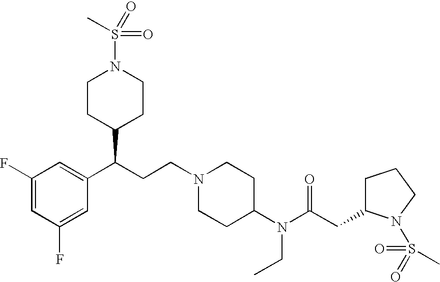

### Step 1: Preparation of tert-butyl (2S)-2-{2-[(1-{(3R)-3-(3,5-difluorophenyl)-3-[1-(methylsulfonyl)piperidin-4-yl]propyl}piperidin-4-yl)(ethyl)amino]-2-oxoethyl}pyrrolidine-1-carboxylate

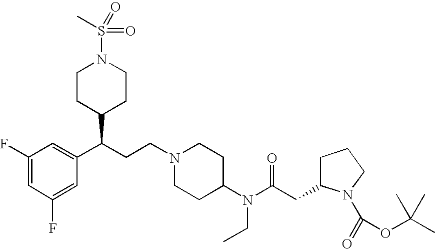

To a solution of [(2S)-1-(tert-butoxycarbonyl)pyrrolidin-2-yl]acetic acid (399 mg) and HATU (343 mg) in DMF (10 ml) was added triethylamine (182 mg) and the resulting mixture was stirred at room temperature for 10 minutes. 1-{(3R)-3-(3,5-difluorophenyl)-3-[1-(methylsulfonyl)piperidin-4-yl]propyl}-N-ethylpiperidin-4-amine was added and the reaction mixture was stirred at room temperature for 24 hours. The reaction mixture was washed with 2N NaOH (2×10 ml), dried over MgSO₄ and evaporated. The residue was purified by silica chromatography eluting with a gradient of ethyl acetate to 20% methanol/ethyl acetate to yield the subtitled compound as a gum (500 mg). LC-MS M+H 655, ¹H NMR (CDCl₃): 1-1.3 (m, 8H) 1.4 (s, 9H) 1.45-2.1 (m, 18H) 2.6 (m, 3H) 2.7 (s, 3H) 2.7-2.9 (m, 2H) 3.0-3.4 (m, 4H) 3.65 (d, 1H) 3.8 (d, 1H) 6.6 (m, 3H).

### Step 2: Preparation of N-(1-{(3R)-3-(3,5-difluorophenyl)-3-[1-(methylsulfonyl)piperidin-4-yl]propyl}piperidin-4-yl)-N-ethyl-2-[(2S)-pyrrolidin-2-yl]acetamide

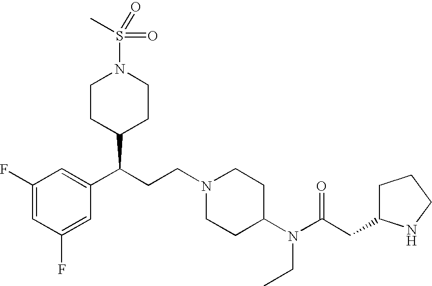

tert-butyl (2S)-2-{2-[(1-{(3R)-3-(3,5-difluorophenyl)-3-[1-(methylsulfonyl)piperidin-4-yl]propyl}piperidin-4-yl)(ethyl)amino]-2-oxoethyl}pyrrolidine-1-carboxylate (400 mg) was dissolved in TFA (10 ml) and stood at room temperature for 1 hour. The TFA was evaporated and the residue dissolved in dichloromethane (50 ml) and washed with 2N NaOH, dried over MgSO₄ and evaporated to yield the subtitled compound (350 mg) as a glass which was used without further purification. LC-MS M+H 555, ¹H NMR (CDCl₃): 1.0-2.1 (m, 23H) 2.2-2.6 (m 7H) 2.7 (s, 3H) 2.75-2.95 (m, 2H) 3.2 (m, 2H) 3.4 (m 1H) 3.65 (d, 1H) 3.8 (d, 1H) 6.6 (m, 3H).

### Step 3: Preparation of Title Compound

N-(1-{(3R)-3-(3,5-difluorophenyl)-3-[1-(methylsulfonyl)piperidin-4-yl]propyl}piperidin-4-yl)-N-ethyl-2-[(2S)-pyrrolidin-2-yl]acetamide (250 mg) was dissolved in dichloromethane (20 ml) and triethylamine (45 mg) was added. Methane sulfonyl chloride (51 mg) was added and the reaction mixture was stirred at room temperature for 2 hours. The reaction mixture was washed with 2N NaOH (2×20 ml), dried over MgSO₄ and evaporated. The residue was dissolved in dichloromethane (10 ml) and poured onto a 20 g SCX2 cartridge and eluted with methanol (6×20 ml) and 1M NH3/methanol (6×20 ml). The combined ammonia washings were evaporated to yield the titled compound as a glass (112 mg). LC-MS M+H 633, ¹H NMR (CDCl₃): 1.0-2.0 (m, 19H) 2.6 (m, 6H) 2.65 (s, 3H) 2.7 (s, 3H) 2.75-3.2 (m, 3H) (m, 3H) 3.3-3.6 (m, 3H) 3.65 (d, 1H) 3.8 (d, 1H) 3.95 (m, 1H) 6.6 (m, 3H).

### EXAMPLE 17

This Example describes the preparation N-(1-{(3R)-3-(3,5-difluorophenyl)-3-[1-(methylsulfonyl)piperidin-4-yl]propyl}piperidin-4-yl)-N-ethyl-3-[1-(methylsulfonyl)piperidin-4-yl]propanamide

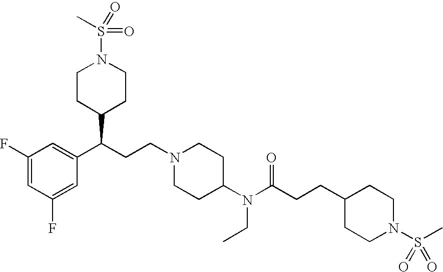

### Step 1: Preparation of (2E)-N-(1-{(3R)-3-(3,5-difluorophenyl)-3-[1-(methylsulfonyl)piperidin-4-yl]propyl}piperidin-4-yl)-N-ethyl-3-[1-(methylsulfonyl)piperidin-4-yl]acrylamide

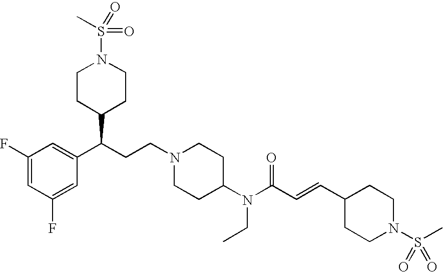

(2E)-3-[1-(methylsulfonyl)piperidin-4-yl]acrylic acid (181 mg) and HATU (342 mg) were dissolved in DMF (10 ml) and triethylamine (91 mg) added. The reaction was stirred at room temperature for 20 minutes. {(3R)-3-(3,5-difluorophenyl)-3-[1-(methylsulfonyl)piperidin-4-yl]propyl}-N-ethylpiperidin-4-amine (Method I) was added and the reaction stirred at room temperature for 24 hours. The solvent was evaporated and the residue dissolved in dichloromethane (50 ml) and washed with 2N NaOH, dried over MgSO4 and evaporated. The residue was purified by silica chromatography eluting with a gradient of ethyl aceate to 25% methanol/ethyl acetate to give the subtitled compound as a glass (yield 420 mg). LC-MS M+H 659, ¹H NMR (CDCl₃): 1.1-2.1 (m, 22H) 2.2-2.6 (m, 7H) 2.65 (s, 3H) 2.7 (m, 3H) 2.8 (m, 1H) 3.2-3.4 (m, 3H) 3.6-3.9 (m, 4H) 6.1 (d, 1H) 6.6 (m, 3H) 6.8 (m, 1H).

### Step 2: Preparation of Title Compound

(2E)-N-(1-{(3R)-3-(3,5-difluorophenyl)-3-[1-(methylsulfonyl)piperidin-4-yl]propyl}piperidin-4-yl)-N-ethyl-3-[1-(methylsulfonyl)piperidin-4-yl]acrylamide (300 mg) was dissolved in ethanol (25 ml) and purged with argon, 20% palladium hydroxide (50 mg) was added and the reaction purged with argon and filled with hydrogen from a balloon. The reaction mixture was stirred at room temperature under a hydrogen atmosphere for 18 hours. The reaction mixture was purged with argon and filtered through celite and evaporated to give the title compound as a solid (yield 214 mg). LC-MS M+H 661, ¹H NMR (CDCl₃): 1.1-2.6 (m, 33H) 2.65 (s, 3H) 2.7 (s, 3H) 2.8-2.9 (m, 2H) 3.2 (m, 2H) 3.6-3.8 (m, 4H) 6.6 (m, 3H)

### Method A

### Preparation of (3R)-3-(3,5-difluorophenyl)-3-[1-(methylsulfonyl)piperidin-4-yl]propanal

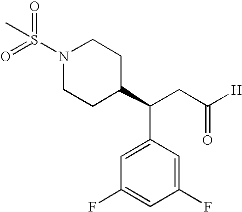

### Step 1 Preparation of (2E)-3-[1-(methylsulfonyl)piperidin-4-yl]acryloyl chloride

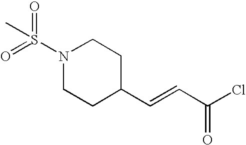

Oxalyl chloride (5.1 g) was added to a solution of (2E)-3-[1-(methylsulfonyl)piperidin-4-yl]acrylic acid (9.4 g) in dichloromethane containing 2-3 drops of DMF and the mixture was stirred at room temperature for 1.5 hours. The reaction mixture was evaporated to dryness and the residue obtained was used directly in the next step.

### Step 2 Preparation of (4R,5S)-1,5-dimethyl-3-{(2E)-3-[1-(methylsulfonyl)piperidin-4-yl]prop-2-enoyl}-4-phenylimidazolidin-2-one

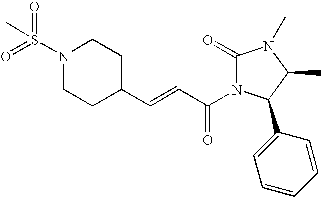

Lithium bis(trimethylsilyl)amide (8 ml of a 1M solution in THF) was added dropwise to a suspension of (4R,5S)-1,5-dimethyl-4-phenyl-2-imidazolidinone (1.52 g) in THF (20 ml) under argon at −10° C. The reaction mixture was stirred at −10° C. for 10 minutes, allowed to warm to 0° C. and maintained at this temperature for 10 minutes then cooled again to −10° C. The solution of the acid chloride (2 g dissolved in 10 ml of dichloromethane) prepared in Step 1 was added dropwise and the reaction mixture was allowed to warm to room temperature and washed with water (100 ml). The aqueous extract was extracted with ethyl acetate (3×50 ml) and the ethyl acetate extracts were dried and the residue passed through a 90 g Biotage column eluting with a solvent gradient (50% ethyl acetate/isohexane-70% ethyl acetate/isohexane). Yield 1.89 g. LC-MS MH⁺ 406, NMR (CDCl₃): 0.8 (d, 3H), 1.5-1.6 (m, 3H), 1.9 (m, 2H), 2.3 (m, 1H), 2.7 (m, 2H), 2.75 (s, 3H), 2.8 (s, 3H), 3.75 (m, 2H), 3.9 (m, 1H), 5.3 (d, 1H), 6.85 (d-d, 1H), 7.1 (d, 1H), 7.2-7.35 (m, 3H), 7.45 (d, 1H).

### Step 3 Preparation of (4S,5R)-1-{(3R)-3-(3,5-difluorophenyl)-3-[1-(methylsulfonyl)piperidin-4-yl]propanoyl}-3,4-dimethyl-5-phenylimidazolidin-2-one

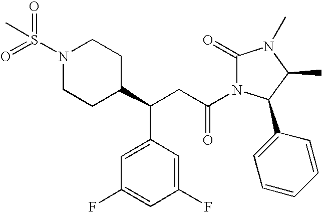

### Step A

TMEDA (11.6 g) was added to a suspension of copper iodide (19.4 g) in THF (240 ml) under argon and the mixture was stirred for 45 minutes then cooled to −70° C. A solution of 3,5-difluorophenyl magnesium bromide in THF (201.1 ml of a 0.5M solution in THF) was added over 10 minutes and the mixture was stirred at −70° C. for 30 minutes.

### Step B

Di-n-butylboron triflate (100.7 ml of 1M solution in dichloromethane) was added to a suspension of (4R,5S)-1,5-dimethyl-3-{(2E)-3-[1-(methylsulfonyl)piperidin-4-yl]prop-2-enoyl}-4-phenylimidazolidin-2-one (20.41 g) [Step 2] in THF maintained at −40° C. and stirring was continued for 10 minutes and the mixture was cooled to −70° C. and added via a cannula to the cuprate suspension prepared in step A. The reaction mixture was stirred at −70° C. for 1 hour and allowed to warm to room temperature, then saturated ammonium chloride solution (200 ml) was added. The THF was evaporated and ethyl acetate (200 ml) was added. Air was blown through this mixture for 1 hour. The ethyl acetate layer was collected and the aqueous portion was extracted with ethyl acetate (2×100 ml). The combined ethyl acetate extracts were washed with saturated ammonium chloride solution (2×100 ml), dried and evaporated to dryness. The residue was purified by chromatography on silica eluting with a solvent gradient of ethyl acetate-isohexane (1:1) to neat ethyl acetate to give the subtitled compound as a white solid, yield 25 g, NMR (CDCl₃) 0.78 (d, 3H), 1.2-1.6 (m, 6H), 1.9 (m, 1H), 2.4-2.65 (m, 2H), 2.75 (s, 3H), 2.85 (s, 3H), 3-3.2 (m, 2H), 3.7-3.9 (m, 4H), 5.2 (d, 1H), 6.6 (m, 3H), 6.85 (m, 2H), 7.2 (m, 3H).

### Step 4 Preparation of (3R)-3-(3,5-difluorophenyl)-3-[1-(methylsulfonyl)piperidin-4-yl]propan-1-ol

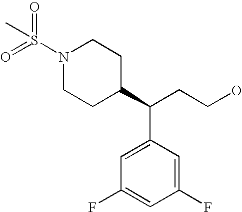

Lithium borohydride (48 ml of 2M solution in THF) was added to a solution of (4S,5R)-1-{(3R)-3-(3,5-difluorophenyl)-3-[1-(methylsulfonyl)piperidin-4-yl]propanoyl}-3,4-dimethyl-5-phenylimidazolidin-2-one (25 g) in THF (200 ml) and the mixture was heated at 70° C. for 3 hours then allowed to cool to room temperature and stirring was continued for 16 hours. Ethanol was added carefully (20 ml) and the reaction mixture was acidified to pH 4 by addition of 2M HCl. The THF was evaporated and the residue dissolved in dichloromethane (100 ml) and this was washed with water (100 ml) and dried. The solvent was removed and the product was purified by chromatography on a Biotage 65 column eluted with a 1:1 mixture of ethyl acetate/isohexane. Yield 13 g, NMR (CDCl₃): 1.2-1.8 (m, 5H), 1.95-2.2 (m, 2H), 2.5-2.7 (m, 3H), 2.75 (s, 3H), 3.3-3.6 (m, 2H), 3.7-3.9 (m, 2H), 6.65 (m, 3H).

### Step 5 Preparation of Title Compound

Dess-Martin periodinane (5.09 g) was added to a solution of (R) 3-(N-methanesulphonylpiperidin-4-yl)-3-(3,5-difluorophenyl)propanol (4.0 g) in dichloromethane (100 ml) and the mixture was stirred for 1.5 hours. The reaction mixture was washed with 2M NaOH (2×100 ml) and dried. The solution of the title compound in dichloromethane was used in subsequent reactions.

In an analogous manner but using 3-(N-trifluoromethylsulphonylpiperidin-4-yl)propenoic acid instead of 3-(N-methanesulphonylpiperidin-4-yl)propenoic acid in step 1 was prepared (R) 3-(N-trifluoromethylsulphonylpiperidin-4-yl)-3-(3,5-difluorophenyl)propionaldehyde.

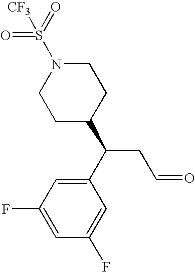

In an analogous manner but using 3-fluorophenyl magnesium bromide instead of 3,5-difluorophenyl magnesium bromide in Step 3 was prepared (3R)-3-(3-fluorophenyl)-3-[1-(methylsulfonyl)piperidin-4-yl]propanal

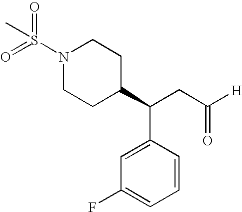

### Method B

### Preparation of tert-butyl 4-{2-[ethyl(piperidin-4-yl)amino]-2-oxoethyl}piperazine-1-carboxylate

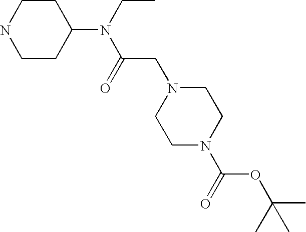

### Step 1: Preparation of tert-butyl 4-{2-[{1-[(benzyloxy)carbonyl]piperidin-4-yl} (ethyl)amino]-2-oxoethyl}piperazine-1-carboxylate

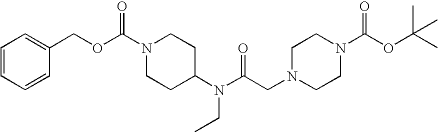

Diisopropylethylamine (1.3 ml) was added to a slurry of [4-(tert-butoxycarbonyl)piperazin-1-yl]acetic acid dihydrate (1.12 g) [154478-71-6] in dichloromethane (16 ml) followed by HATU (1.82 g) and the mixture was stirred under argon for 30 minutes. A solution of benzyl 4-(ethylamino)piperidine-1-carboxylate (1.05 g) [159874-38-1] in dichloromethane (4 ml) was added and the mixture was stirred for 24 hours, then diluted with dichloromethane (25 ml), washed consecutively with 2M NaOH (2×20 ml) and brine (1×20 ml) and dried. The solvent was evaporated and the residue was purified on a 40 g silica column eluted with a solvent gradient made up of ethyl acetate to 5% methanol:ethyl acetate. The yellow oil obtained was used directly in the next stage, LC-MS M+H 489 plus a HATU derived impurity M+H 175.

¹H NMR (CDCl₃): 1.12 (3H, m), 1.44 (9H, s), 1.54-1.74 (8H, m), 2.48 (4H, d), 3.19-3.38 (4H, m), 3.44 (4H, d), 4.30 (1H, m), 5.14 (2H, s). 7.36 (5H, s).

### Step 2: Preparation of the Title Compound

A solution of tert-butyl 4-{2-[{1-[(benzyloxy)carbonyl]piperidin-4-yl}(ethyl)amino]-2-oxoethyl}piperazine-1-carboxylate (1.98 g) in ethanol (20 ml) was hydrogenated under a hydrogen filled balloon using 20% Pd(OH)₂ on carbon as catalyst. The reaction mixture was filtered through Celite and the solvent was evaporated under reduced pressure. The residue was purified by passing down a SCX-2 silica column eluting with methanol initially and then with a 1M solution of ammonia in methanol. The methanolic ammonia fractions were evaporated to dryness to give the title compound as an oil, yield 1.3 g, LC-MS M+H 335. ¹H NMR (CDCl₃): 1.18 (3H, m), 1.44 (9H, s), 1.72 (8H, m), 2.48 (3H, m), 2.70 (1H, m), 3.22 (2H, m), 3.32 (2H, m), 3.46 (4H, m), 3.92 & 4.38 (1H, m).

In an analogous manner but using benzyl 4-(isobutylamino)piperidine-1-carboxylate instead of benzyl 4-(ethylamino)piperidine-1-carboxylate in Step 1 was prepared tert-butyl 4-{2-[isobutyl(piperidin-4-yl)amino]-2-oxoethyl}piperazine-1-carboxylate

In an analogous manner but using benzyl 4-(methylamino)piperidine-1-carboxylate instead of benzyl 4-(ethylamino)piperidine-1-carboxylate in Step 1 was prepared tert-butyl 4-{2-[isobutyl(piperidin-4-yl)amino]-2-oxoethyl}piperazine-1-carboxylate

### Method C

### Preparation of tert-butyl 4-{2-[ethyl(piperidin-4-yl)amino]-2-oxoethyl}piperidine-1-carboxylate

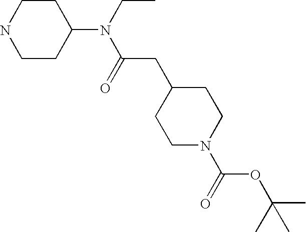

### Step 1: Preparation of tert-butyl 4-{2-[{1-[(benzyloxy)carbonyl]piperidin-4-yl} (ethyl)amino]-2-oxoethyl}piperidine-1-carboxylate

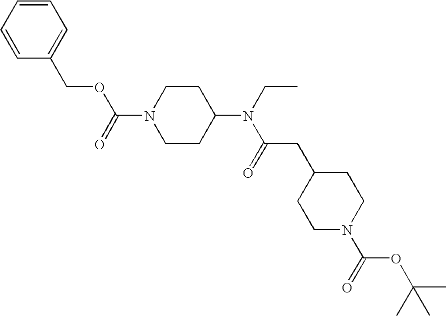

1-Chloro-N,N-2-trimethylpropenylamine (891 μl) was added to a solution of [1-(tert-butoxycarbonyl)piperidin-4-yl]acetic acid (1.60 g) [157688-46-5] in dichloromethane (10 ml) and the mixture was stirred for 2 hours. An additional equivalent of 1-chloro-N,N-2-trimethylpropenylamine was added and stirring continued for 3 hours. A solution of benzyl 4-(ethylamino)piperidine-1-carboxylate (1.735 g) and triethylamine (1.84 ml) in dichloromethane (30 ml) was added and the mixture was stirred for 16 hours then diluted with dichloromethane (30 ml) and washed with saturated ammonium chloride solution (2×25 ml), 2M NaOH (2×25 ml) and brine (25 ml) and dried. Removal of the solvent gave an orange oil, LC-MS M+H (-Boc) 388. The material was used directly in step 2.

### Step 2: Preparation of the Title Compound

A solution of tert-butyl 4-{2-[{1-[(benzyloxy)carbonyl]piperidin-4-yl}(ethyl)amino]-2-oxoethyl}piperidine-1-carboxylate (3.1 g) in ethanol (30 ml) was hydrogenated under a hydrogen filled balloon using 20% Pd(OH)₂ on carbon as catalyst. The reaction mixture was filtered through Celite and the solvent was evaporated under reduced pressure. The residue was purified by passing down a SCX-2 50 g silica column eluting with methanol initially and then with a 1M solution of ammonia in methanol. The methanolic ammonia fractions were evaporated to dryness to give the title compound as an oil, yield 2.08 g, ¹H NMR (CDCl₃): 1.14 (3H, m), 1.44 (9H, s), 1.52-1.78 (8H, m), 2.10 (1H, m), 2.22 (2H, m), 2.70 (4H, m), 3.14 (2H, m), 3.28 (2H, m), 3.62 & 4.48 (1H, m), 4.10 (2H, m).

Following the procedures outlined above and using benzyl 4-(ethylamino)piperidine-1-carboxylate and [1-(tert-butoxycarbonyl)piperidin-4-yl]acetic acid [157688-46-5] as starting materials there is obtained benzyl 4-[{[4-(tert-butoxycarbonyl)piperidin-1-yl]acetyl}(ethyl)amino]piperidine-1-carboxylate.

### Method D

### Preparation of N-ethyl-N′-{[1-(methylsulfonyl)piperidin-4-yl]methyl}-N-piperidin-4-ylurea

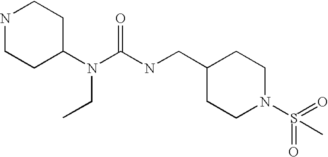

### Step 1. Preparation of 2,2,2-trichloro-N-{[1-(methylsulfonyl)piperidin-4-yl]methyl}acetamide

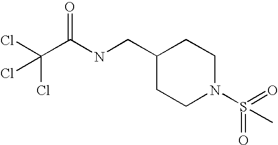

Trichloroacetyl chloride (0.41 ml) was added to a solution of {[1-(methylsulfonyl)piperidin-4-yl]methyl}amine (700 mg) [325153-03-5] in dichloromethane (20 ml) containing pyridine (0.59 ml) and DMAP (73 mg) and the mixture was stirred for 3 hours then washed with water (1×15 ml), brine (1×15 ml) and dried. The residue obtained on removal of the solvent was purified on a 20 g silica Bond Elut eluted with a solvent gradient of 1:1 ethyl acetate/hexane to ethyl acetate to give 2,2,2-trichloro-N-{[1-(methylsulfonyl)piperidin-4-yl]methyl}acetamide, yield 578 mg, LC-MS M+H 337/339.

¹H NMR (CDCl₃): 1.3-1.5 (m, 2H) 1.8 (m, 3H) 2.7 (m, 3H) 3.3 (m, 2H) 3.8 (m, 2H) 6.8 (bs, 1H).

### Step 2: Preparation of tert-butyl 4-{ethyl[({[1-(methylsulfonyl)piperidin-4-yl]methyl}amino)carbonyl]amino}piperidine-1-carboxylate

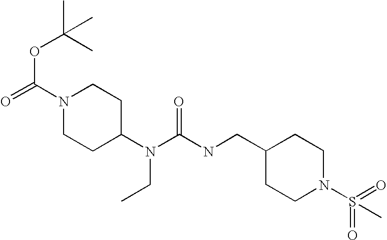

A solution of 2,2,2-trichloro-N-{[1-(methylsulfonyl)piperidin-4-yl]methyl}acetamide (578 mgs) in DMA (3 ml) was added to a stirred solution of tert-butyl 4-(ethylamino)piperidine-1-carboxylate (521 mg) in DMA (7 ml) followed by DBU (0.34 ml) and the mixture was stirred at 85° C. for 4 hours. The solvent was evaporated and the residue was purified on a 20 g silica Bond Elut eluted with a solvent gradient of 1:1 ethyl acetate/hexane to ethyl acetate. Yield 770 mg, LC-MS M+H 469.

¹H NMR (CDCl₃): 1.2 (m, 3H) 1.4 (m, 2H) 1.5 (s, 9H) 1.6 (m, 3H) 1.8 (m, 1H) 2.1 (m, 3H) 2.6-2.8 (m, 5H) 2.9 (m, 3H) 3.0 (s, 3H) 3.1-3.2 (m, 3H) 3.8 (m, 1H) 4.2 (m, 1H).

### Step 3: Preparation of the Title Compound

TFA (5 ml) was added to a solution of tert-butyl 4-{ethyl[({[1-(methylsulfonyl)piperidin-4-yl]methyl}amino)carbonyl]amino}piperidine-1-carboxylate (750 mg) in dichloromethane (20 ml) and the mixture was stirred for 1 hour. The solvent was evaporated and the residue was redissolved in 2M NaOH (1×15 ml) and extracted with dichloromethane (2×15 ml). The dichloromethane extracts were dried and the solvent removed to give the title compound, yield 480 mg, LC-MS M+H 347.

### Method E

### Preparation of N-allyl-2-[1-(methylsulfonyl)piperidin-4-yl]-N-piperidin-4-ylacetamide hydrochloride

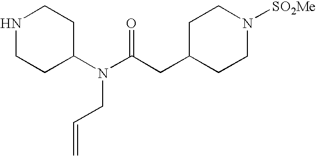

### Step 1: Preparation of tert-butyl 4-(allyl{[1-(methylsulfonyl)piperidin-4-yl]acetyl}amino)piperidine-1-carboxylate

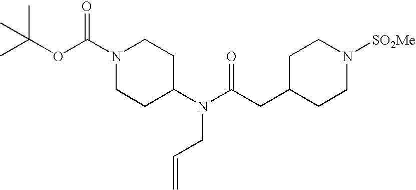

To a solution of [1-(methylsulfonyl)piperidin-4-yl]acetic acid [CAS 423722-27-4] (0.8 g) in CH₂Cl₂ (20 ml) was added 1-chloro-N,N-2-trimethylpropenylamine (0.53 ml) and the mixture stirred at room temperature for 2 h. A solution of tert-butyl 4-(allylamino)piperidine-1-carboxylate [CAS 235420-68-5] (770 mg) and triethylamine (1 ml) in CH₂Cl₂ (10 ml) was then added and the mixture stirred at room temperature for 18 h. The reaction mixture was poured into aqueous NaHCO₃ (50 ml) and extracted with CH₂Cl₂ (2×100 ml). The combined extracts were washed with brine (100 ml) and dried (MgSO₄). Concentration under reduced pressure gave the crude product as a clear oil (1.5 g), M+H⁺ (344, M⁺-BOC).

NMR (CDCl₃): 1.2-1.5 (m, 3H), 1.4 (s, 9H), 1.6 (d, 2H), 1.8-1.9 (m, 4H), 2.6-2.8 (m, 3H), 2.8 (s, 3H), 3.7-3.8 (m, 4H), 4.1-4.3 (m, 2H), 5.1-5.3 (m, 2H), 5.7-5.9 (m, 1H).

### Step 2: Preparation of the Title Compound

A solution of tert-butyl 4-(allyl{[1-(methylsulfonyl)piperidin-4-yl]acetyl}amino)piperidine-1-carboxylate (800 mg) in CH₂Cl₂ (20 ml) and saturated methanolic hydrogen chloride (15 ml) was stirred at room temperature for 18 h. The reaction mixture was concentrated under reduced pressure to give the crude product as a hygroscopic white solid (560 mg). M+H⁺ (344.3).

NMR (DMSO): 1.1-1.3 (m, 2H), 1.5-2.1 (m, 8H), 2.2 (d, 1H), 2.4 (d, 1H), 2.7 (m, 3H), 2.8 (s, 3H), 2.9-3.0 (m, 3H), 3.3 (d, 2H), 3.9 (br m, 2H), 4.5 (m, 1H), 5.0-5.3 (m, 2H), 5.8-5.9 (m, 1H).

### Method F

### Preparation of 1-{(3R)-3-(3,5-difluorophenyl)-3-[4-(methylsulphonyl)phenyl]propyl}-N-ethylpiperidin-4-amine

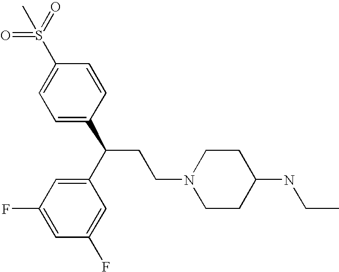

Sodium triacetoxyborohydride (2.54 g) was added in portions to a solution of (R)-3-(3,5-difluorophenyl)-3-[4-(methylsulphonyl)phenyl]propanal in dichloromethane (3.2 g in 50 ml, Method G) and tert-butyl ethyl(piperidin-4-yl)carbamate (2.28 g) [CAS 313977-45-6] in dichloromethane (50 ml) containing acetic acid (0.2 ml) and the mixture was stirred for 16 hours. The mixture was diluted with dichloromethane (50 ml) and washed with saturated aqueous sodium bicarbonate (2×25 ml) and brine (25 ml) and dried. The residue obtained on evaporation of the solvent was purified on a silica column eluting with 5% methanol/ethyl acetate, yield 4.18 g, M+H 537.

### Method G

### Preparation of (R)-3-(3,5-difluorophenyl)-3-[4-(methylsulphonyl)phenyl]propanal

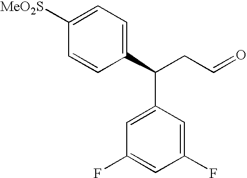

### Step 1: Preparation of E-(4S,5R)-1-(3-[4-methanesulphonylphenyl]acryloyl)-3,4-dimethyl-5-phenyl-imidazolidin-2-one

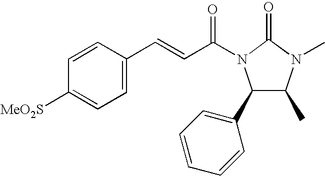

To a stirred solution of 3-(4-methanesulphonylphenyl)acrylic acid (7.14 g) in DCM (10 mL) was added thionyl chloride (3 mL) dropwise and the resulting mixture was stirred at room temperature for 18 h. To this solution was added DIPEA (5.04 mL) dropwise at room temperature. The resulting solution was added to a stirred solution of (4R,5S)-1,5-dimethyl-4-phenyl-imidazolidin-2-one (5.0 g) in DCM (20 mL) and DIPEA (4.58 mL) and the resulting mixture stirred at room temperature for 4 h. The mixture was washed with water and brine, pre-absorbed onto a Bond Elut and eluted with a gradient of isohexane to ethyl acetate giving the sub-titled compound as a solid (7.61 g, 73%).

NMR (CDCl₃): 0.84 (d, 3H), 2.89 (s, 3H), 3.04 (s, 3H), 3.98 (m, 1H), 5.42 (d, 1H), 7.20 (m, 2H), 7.32 (m, 3H), 7.69 (d, 1H), 7.74 (d, 2H), 7.93 (d, 2H), 8.31 (d, 1H); MS: 399.

### Step 2: Preparation of (4S,5R)-1-[(R)-3-(4-methanesulphonyl-phenyl)-3-(3,5-di-fluorophenyl)-propionyl]-3,4-dimethyl-5-phenyl-imidazolidin-2-one

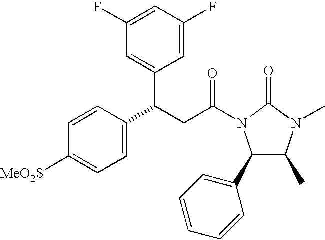

To a mixture of copper (I) iodide (5.01 g) and THF (90 mL) was added N,N,N′,N′-tetramethylethylenediamine (4.2 mL) and the resulting mixture was stirred at room temperature for 10 min. then cooled to −78° C. 3,5-Difluorophenylmagnesium bromide (52 mL, 0.5M in THF) was added and the resulting mixture stirred at −78° C. for 30 min. A solution of di-n-butylboron triflate (15.8 mL, 1M in diethyl ether) and (E)-(4S,5R)-1-(3-[4-methanesulphonylphenyl]acryloyl)-3,4-dimethyl-5-phenyl-imidazolidin-2-one (5.2 g) in THF (90 mL) was added gradually and the resulting mixture was stirred whilst allowing to warm to room temperature for 18 h. The reaction mixture was washed with saturated aqueous ammonium chloride then concentrated tetrasodium EDTA solution and evaporated to give a yellow solid. This was triturated with diethyl ether giving the sub-titled compound (4.04 g, 60%) as a white powder.

NMR: 0.78 (d, 3H), 2.83 (s, 3H), 3.26 (s, 3H), 3.75 (dd, 1H), 4.05 (m, 2H), 4.80 (t, 1H), 5.35 (d, 1H), 7.10 (m, 3H), 7.20 (m, 2H), 7.35 (m, 3H), 7.73 (d, 2H), 7.93 (d, 2H); LC-MS: 513.

### Step 3: Preparation of (R)-3-(3,5-difluorophenyl)-3-[4-(methylsulphonyl)phenyl]propanol

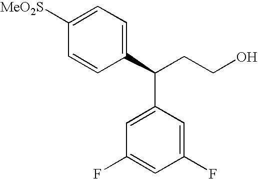

To a mixture of (4S,5R)-1-[(R)-3-(4-methanesulphonyl-phenyl)-3-(3,5-difluorophenyl)-propionyl]-3,4-dimethyl-5-phenyl-imidazolidin-2-one (57 g) and THF (500 mL) at 20° C. was added lithium borohydride (2M in THF, 80 mL) gradually. The resulting mixture was heated to reflux for 1 h, cooled to 5° C. and the reaction quenched by the gradual addition of 2M hydrochloric acid (200 mL). The mixture was extracted with diethyl ether and the extracts dried and concentrated. The residue was triturated with ethyl acetate (200 mL) and the resulting mixture filtered. The filtrate was concentrated and purified by silica column chromatography (eluting with ethyl acetate) to give the sub-titled compound as an oil (25.5 g).

NMR (CDCl₃): 1.65 (br s, 1H), 2.3 (m, 2H), 3.55 (m, 2H), 4.3 (t, 1H), 6.7 (m, 1H), 6.75 (m, 2H), 7.25 (d, 2H), 7.9 (d, 2H).

### Step 4: Preparation of the Title Compound

Dess-Martin periodinane (5.09 g) was added to a solution of (R)-3-(3,5-difluorophenyl)-3-[4-(methylsulphonyl)phenyl]propanol (3.26 g) in dichloromethane (50 ml) and the mixture was stirred for 45 minutes, diluted with an equal volume of dichloromethane and washed with 2M NaOH (2×25 ml) and brine (25 ml) and dried. The solution obtained on filtration of the drying agent was used directly in subsequent steps.

### Method H

### Preparation of N-(cyclopropylmethyl)-N-(1-{(3R)-3-(3,5-difluorophenyl)-3-[1-(methylsulfonyl)piperidin-4-yl]propyl}piperidin-4-yl)-2-piperazin-1-ylacetamide

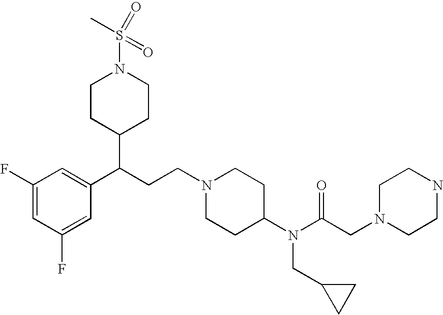

### Step 1: Preparation of benzyl 4-[(cyclopropylmethyl)amino]piperidine-1-carboxylate

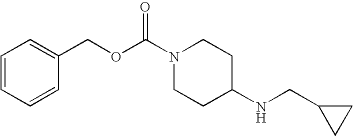

To a solution of benzyl-4-oxopiperidine-1-carboxylate (10 g) in dichloromethane (250 ml) was added (cyclopropylmethyl)amine (6.09 g) and the resulting mixture was stirred at room temperature for 30 minutes. The reaction mixture was cooled to 0° C. and sodium triacetoxyborohydride (10.9 g) was added. The mixture was allowed to reach room temperature and then stirred for 18 hours. The reaction was quenched with 2N NaOH (100 ml). The organic phase was dried over MgSO₄, filtered and evaporated to dryness to yield an oil, which was vacuum dried. (Yield 12.1 g).

CDCl3: 0.1 (m, 2H), 0.6 (m, 2H), 0.85 (m, 1H), 1.15 (br, 3H), 1.7 (br, 2H), 2.4 (m, 2H), 2.52 (m, 1H), 2.89 (br, 2H), 4.0 (br, 2H), 5.02 (s, 2H), 7.25 (m, 5H).

### Step 2: Preparation of benzyl 4-[(tert-butoxycarbonyl)(cyclopropylmethyl)amino]piperidine-1-carboxylate

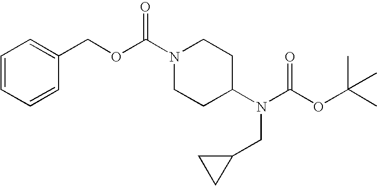

To a solution of benzyl 4-[(cyclopropylmethyl)amino]piperidine-1-carboxylate (12.05 g) and triethylamine (6.98 ml) in dichloromethane (250 ml) was added di tert-butyl dicarbonate (10.9 g, in 3 portions over 20 minutes. The reaction mixture was stirred at room temperature for 18 hours. The reaction mixture was washed with 2N NaOH (2×100 ml), dried over MgSO₄, filtered and evaporated to dryness to give an oil which was used without further purification. Yield 17 g. LC-MS (M+H-Boc)=288

### Step 3: Preparation of tert-butyl (cyclopropylmethyl)piperidin-4-ylcarbamate

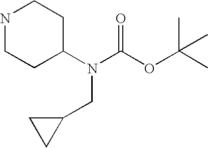

To a solution of benzyl 4-[(tert-butoxycarbonyl)(cyclopropylmethyl)amino]piperidine-1-carboxylate (17 g) in ethanol (200 ml) was added 20% palladium hydroxide on carbon (1.7 g) and the resulting reaction mixture was stirred under a hydrogen atmosphere for 18 hours. The reaction mixture was filtered through Celite and evaporated to dryness to yield an oil which was used without further purification. Yield 10.79 g.

NMR CDCl₃: 0.25 (m, 2H), 0.45 (m, 2H), 0.95 (m, 1H), 1.45 (s, 9H), 1.6 (m, 1H), 1.7 (m, 2H), 1.85 (m, 2H), 2.65 (m, 2H), 3.0 (m, 2H), 3.1 (m, 2H),

### Step 4: Preparation of tert-butyl (cyclopropylmethyl)(1-{(3R)-3-(3,5-difluorophenyl)-3-[1-(methylsulfonyl)piperidin-4-yl]propyl}piperidin-4-yl)carbamate

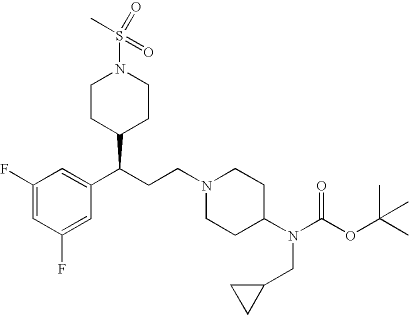

A solution of (3R)-3-(3,5-difluorophenyl)-3-[1-(methylsulfonyl)piperidin-4-yl]propanal (6.6 g) in dichloromethane (100 ml) was added to a solution of tert-butyl (cyclopropylmethyl)piperidin-4-ylcarbamate (5.08 g) in dichloromethane (100 ml). Sodium triacetoxyborohydride (5.09 g) was added and the resulting reaction mixture was stirred at room temperature for 18 hours. The reaction mixture was washed with 2M NaOH (2×150 ml), dried over MgSO₄ and filtered. PS-isocyanate resin (1.2 mm/g; 5 g) was added to the filtrate and the mixture was stirred at room temperature for 3 hours. The reaction mixture was filtered and evaporated to dryness to yield a colourless oil which was used without further purification. Yield 12.4 g. LC-MS (M+H-Boc)=570.

NMR CDCl₃: 0.25 (m, 2H), 0.5 (m, 2H), 0.95 (m, 1H), 1.2-1.4 (m, 4H), 1.45 (s, 9H), 1.6-2.6 (m, 17H), 2.75 (s, 3H), 2.95 (br, 3H), 3.7 (d, 1H), 3.85 (d, 1H), 6.65 (m, 3H).

### Step 5: Preparation of N-(cyclopropylmethyl)-1-{(3R)-3-(3,5-difluorophenyl)-3-[1-(methylsulfonyl)piperidin-4-yl]propyl}piperidin-4-amine

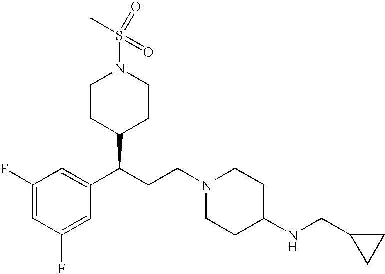

tert-butyl (cyclopropylmethyl)(1-{(3R)-3-(3,5-difluorophenyl)-3-[1-(methylsulfonyl)piperidin-4-yl]propyl}piperidin-4-yl)carbamate (11.4 g) was dissolved in TFA (150 ml) and stirred at room temperature for 1 hour. The reaction mixture was concentrated and the residue was partitioned between dichloromethane (150 ml) and 2NaOH (150 ml). The aqueous layer was extracted with further dichloromethane (3×100 ml). The organic extracts were combined, dried (MgSO₄), filtered and evaporated to dryness to yield an oil which was used without further purification. Yield 8.28 g. LC-MS (M+H)=470. NMR CDCl₃ 0.1 (m, 1H), 0.5 (m, 2H), 0.95-2.1 (m, 16H), 2.3-2.7 (m, 8H), 2.75 (s, 3H), 2.8 (m, 1H), 3.65 (d, 1H), 3.8 (d, 1H), 6.65 (m, 3H).

### Step 6: Preparation of tert-butyl 4-{2-[(cyclopropylmethyl)(1-{(3R)-3-(3,5-difluorophenyl)-3-[1-(methylsulfonyl)piperidin-4-yl]propyl}piperidin-4-yl)amino]-2-oxoethyl}piperazine-1-carboxylate

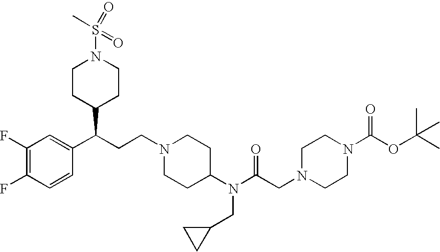

[4-(tert-butoxycarbonyl)piperazin-1-yl]acetic acid (518 mg) and HATU (804 mg) were dissolved in DMF (40 ml) and triethylamine (0.9 ml) was added. The reaction mixture was stirred for 10 minutes at room temperature and then N-(cyclopropylmethyl)-1-{(3R)-3-(3,5-difluorophenyl)-3-[1-(methylsulfonyl)piperidin-4-yl]propyl}piperidin-4-amine (994 mg) was added. The reaction mixture was stirred at room temperature for 24 hours and evaporated to dryness. The residual oil was dissolved in dichloromethane (100 ml), washed with 2M NaOH (2×100 ml), dried over MgSO₄, filtered and evaporated to dryness to yield a yellow oil which was purified by combiflash chromatography using a 12 g Redisep column and eluting with 0-25% MeOH/ethyl acetate gradient to yield an oil. Yield 250 mg. LC-MS (M+H)=696.

NMR CDCl₃ 0.25 (m, 2H), 0.4 (d, 2H), 0.6 (d, 2H), 1.2-1.4 (m, 2H), 1.45 (s, 9H), 1.6 (br, 3H), 1.8 (m, 2H), 2.0 (m, 4H), 2.3-2.7 (m, 10H), 2.75 (s, 3H), 2.90 (m, 2H), 3.05-3.3 (m, 4H), 3.4 (m, 4H), 3.5 (s, 1H), 3.7 (d, 1H), 3.85 (d, 1H), 6.65 (m, 3H).

### Step 7: Preparation of Title Compound

A solution of tert-butyl 4-{2-[(cyclopropylmethyl)(1-{(3R)-3-(3,5-difluorophenyl)-3-[1-(methylsulfonyl)piperidin-4-yl]propyl}piperidin-4-yl)amino]-2-oxoethyl}piperazine-1-carboxylate (231 mg) in TFA (10 ml) was stirred for 1 hour. The reaction mixture was evaporated to dryness and partitioned between 2M NaOH (15 ml) and dichloromethane (3×15 ml). The organic phase was collected, dried over MgSO₄, filtered and evaporated to dryness to yield the title compound as an oil. Yield 195 mg. LC-MS (1+H) 596.

NMR CDCl₃: 0.3 (d, 2H), 0.55 (dd, 2H), 1.3-2.65 (m, 24H), 2.75 (s, 3H), 2.95 (m, 7H), 3.2 (m, 4H), 3.75 (d, 1H), 3.85 (d, 1H), 6.65 (m, 3H).

### Method I

### Preparation of 1-{(3R)-3-(3,5-difluorophenyl)-3-[1-(methylsulfonyl)piperidin-4-yl]propyl}-N-methylpiperidin-4-amine

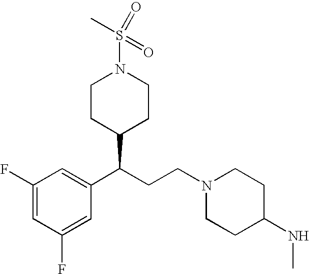

### Step 1: Preparation of tert-butyl (1-{(3R)-3-(3,5-difluorophenyl)-3-[1-(methylsulfonyl)piperidin-4-yl]propyl}piperidin-4-yl)methylcarbamate

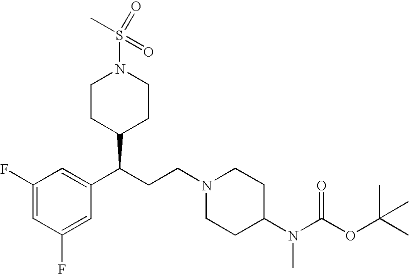

To a solution of tert-butyl methyl(piperidin-4-yl)carbamate (CAS 188174-17-6) (4 g) and (3R)-3-(3,5-difluorophenyl)-3-[1-(methylsulfonyl)piperidin-4-yl]propanal (Method A) (6.2 g) in dichloromethane (100 ml) was added sodium triacetoxyborohydride (4.7 g). The reaction mixture was stirred at room temperature for 24 hours and then washed with 2N NaOH (2×100 ml), dried over MgSO₄ and evaporated. The residue was purified by silica chromatography eluting with a gradient of ethylacetate to 20% methanol/ethyl acetate to yield the sub titled compound as a pale brown oil. Yield 9.7 g LC-MS (M+H) 530. NMR CDCl₃: 1.2-1.35 (m, 3H) 1.4 (s, 9H) 1.6 (m, 7H) 1.9-2.1 (m, 6H) 2.35 (m, 1H) 2.5 (m, 1H) 2.6 (m, 1H) 2.7 (s, 3H) 2.75 (s, 3H) 2.8-2.9 (m, 2H) 3.7 (m, 1H) 3.85 (m, 1H) 6.6 (m, 3H).

### Step 2: Preparation of Title Compound

Trifluoroacetic acid (25 ml) was added to tert-butyl (1-{(3R)-3-(3,5-difluorophenyl)-3-[1-(methylsulfonyl)piperidin-4-yl]propyl}piperidin-4-yl)methylcarbamate (9.7 g) and stirred at room temperature for 1 hour. The trifluoroacetic acid was evaporated off and 2N NaOH (100 ml) added and the mixture extracted with dichloromethane (3×100 ml). The combined extracts were dried over MgSO₄ and evaporated to leave a pale brown gum which was used without further purification. Yield 7.6 g. LC-MS (M+H) 430 NMR CDCl₃: 1.2-2.2 (m, 15H) 2.4 (m, 2H) 2.45 (s, 3H) 2.5-2.7 (m, 3H) 2.75 (s, 3H) 2.8 (m, 1H) 3.7 (m, 1H) 3.85 (m, 1H) 6.65 (m, 3H).

In a similar manner but using tert-butyl isobutyl(piperidin-4-yl)carbamate in Step 1 was prepared 1-{(3R)-3-(3,5-difluorophenyl)-3-[1-(methylsulfonyl)piperidin-4-yl]propyl}-N-isobutylpiperidin-4-amine.

In a similar manner but using tert-butyl ethyl(piperidin-4-yl)carbamate in Step 1 was prepared 1-{(3R)-3-(3,5-difluorophenyl)-3-[1-(methylsulfonyl)piperidin-4-yl]propyl}-N-ethylpiperidin-4-amine

### Method J

### Preparation of [1-(methylsulfonyl)pyrrolidin-3-yl)acetic acid

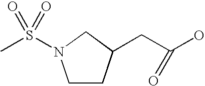

### Step 1: Preparation of tert-butyl [1-(methylsulfonyl)pyrrolidin-3-yl]acetate

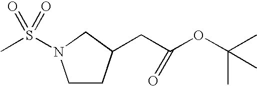

To a solution of tert-butylpyrrolidin-3-yl-acetate (1 g) and triethylamine (0.85 ml) in dichloromethane (50 ml) was added methanesulfonylchloride (0.48 ml). The reaction mixture was stirred at room temperature for 24 hours. The reaction mixture was washed with 2N NaOH (2×20 ml), dried over MgSO₄ and evaporated to yield a mobile oil (yield 1.8 g) which was used without further purification.

### Step 2: Preparation of Title Compound

tert-butyl [1-(methylsulfonyl)pyrrolidin-3-yl]acetate (1.8 g) was dissolved in TFA (20 ml) and stood at room temperature for 1 hour. The TFA was evaporated. The residue was triturated with ether (6×20 ml) and the combined ether extracts was evaporated to yield a white solid (yield 140 mg). LC-MS (M−H) 206 NMR CDCl₃: 1.6 (m, 1H) 2.15 (m, 1H) 2.4 (m, 2H) 2.6 (m, 1H) 2.75 (s, 3H) 2.9 (m, 1H) 3.25 (m, 1H) 3.4 (m, 1H) 3.6 (m, 1H).

### Method K

Preparation of N-(1-{(3R)-3-(3,5-difluorophenyl)-3-[1-(methylsulfonyl)piperidin-4-yl]propyl}piperidin-4-yl)-N-isobutyl-N′-piperidin-4-ylurea

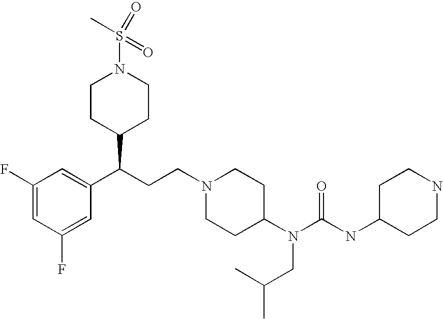

### Step 1: Preparation of benzyl 4-({[(1-{(3R)-3-(3,5-difluorophenyl)-3-[1-(methylsulfonyl)piperidin-4-yl]propyl}piperidin-4-yl)(isobutyl)amino]carbonyl}amino)piperidine-1-carboxylate

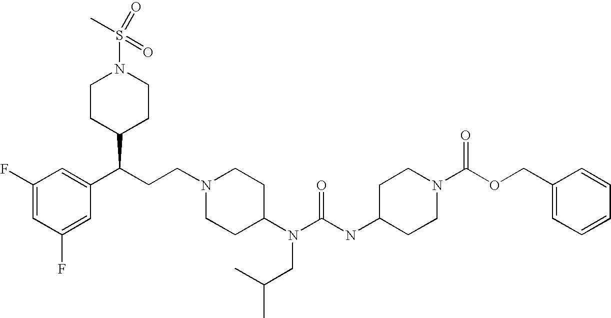

1-{(3R)-3-(3,5-difluorophenyl)-3-[1-(methylsulfonyl)piperidin-4-yl]propyl}-N-isobutylpiperidin-4-amine (Method I) (600 mg) was dissolved in dichloromethane (25 ml) and benzyl 4-isocyanatopiperidine-1-carboxylate (331 mg) added. The reaction mixture was stirred at room temperature for 18 hours. The reaction mixture was poured onto a 20 g SCX2 cartridge and eluted with methanol (6×20 ml) and 1M ammonia/methanol (6×20 ml). The combined ammonia washings were evaporated to a gum which was purified by silica chromatography eluting with a gradient of ethyl acetate to 20% methanol/ethyl acetate to give the subtitled compound as a solid. Yield 750 mg. LC-MS (M+H) 732 NMR CDCl₃: 0.85 (6H, d) 1.2-1.7 (10H, m) 1.8-2.1 (9H, m) 2.35 (1H, m) 2.5 (1H, m) 2.6 (1H, m) 2.7 (3H, s) 2.8-95 (5H, m) 3.7 (1H, m) 3.8 (2H, m) 3.95 (1H, m) 4.05-4.2 (4H, m) 5.2 (2H, s) 6.7 (3H, m) 7.35 (5H, m).

### Step 2: Preparation of Title Compound

Benzyl 4-({[(1-{(3R)-3-(3,5-difluorophenyl)-3-[1-(methylsulfonyl)piperidin-4-yl]propyl}piperidin-4-yl)(isobutyl)amino]carbonyl}amino)piperidine-1-carboxylate (600 mg) was dissolved in ethanol (50 ml) and purged with argon. 20% Palladium hydroxide (100 mg) was added and the reaction purged with argon. Hydrogen was introduced from a balloon and the reaction was stirred under a hydrogen atmosphere for 24 hours. The reaction was purged with argon, filtered through celite and evaporated to give the title compound. Yield 500 mg LC-MS (M+H) 598 NMR CDCl₃: 0.9 (6H, d) 1.1-2.2 (17H, m) 2.4 (1H, m) 2.5 (1H, m) 2.55-2.77 (4H, m) 2.75 (3H, s) 2.8-2.9 (4H, m) 3.05 (2H, m) 3.7-3.8 (3H, m) 3.85 (1H, m) 3.95 (1H, m) 4.25 (1H, m) 6.7 (3H, m).

In an analogous manner but using 1-{(3R)-3-(3,5-difluorophenyl)-3-[1-(methylsulfonyl)piperidin-4-yl]propyl}-N-methylpiperidin-4-amine in Step 1 there was obtained N-(1-{(3R)-3-(3,5-difluorophenyl)-3-[1-(methylsulfonyl)piperidin-4-yl]propyl}piperidin-4-yl)-N-methyl-N′-piperidin-4-ylurea LC-MS (M+H) 556 NMR CDCl₃: 1.2-2.3 (16H, m) 2.35-2.65 (5H, m) 2.7 (3H, s) 2.75 (3H, s) 2.8-2.95 (2H, m) 3.05 (2H, m) 3.7 (4H, m) 3.75 (1H, m) 4.15 (1H, m) 4.2 (1H, d) 6.7 (3H, m).

### EXAMPLE 18

The ability of compounds to inhibit the binding of MIP-1α was assessed by an in vitro radioligand binding assay. Membranes were prepared from Chinese hamster ovary cells which expressed the recombinant human CCR5 receptor. These membranes were incubated with 0.1M iodinated MIP-1α, scintillation proximity beads and various concentrations of the compounds of the invention in 96-well plates. The amount of iodinated MIP-1α bound to the receptor was determined by scintillation counting. Competition curves were obtained for compounds and the concentration of compound which displaced 50% of bound iodinated MIP-1α was calculated (IC₅₀). Preferred compounds of formula (I) have an IC₅₀ of less than 50 μM.

Results from this test for certain compounds of the invention are presented in Table V. In Table V the results are presented as Pic50 values. A Pic50 value is the negative log (to base 10) of the IC₅₀ result, so an IC₅₀ of 1 μM (that is 1×10⁻⁶M) gives a Pic50 of 6. If a compound was tested more than once then the data below is an average of the probative tests results.

TABLE VII

| Compound No. | Table No | Pic50 | Compound No. | Table No | Pic50 |
|---|---|---|---|---|---|
| 5 | I | 8.5 | 1 | III | 9.1 |
| 7 | I | 9.5 | 2 | III | 7.8 |
| 1 | IV | 9.5 | | | |

1. A compound of formula (I): 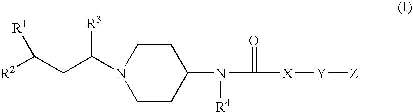
wherein
R¹ is C₁₋₅ alkyl, C(O)NR¹⁰R¹¹, C(O)₂R¹², NR¹³C(O)R¹⁴, NR¹⁵C(O)NR¹⁶R¹⁷, NR¹⁸C(O)₂R¹⁹, heterocyclyl, aryl or heteroaryl;
R¹⁰, R¹³, R¹⁵, R¹⁶ and R¹⁸ are hydrogen or C₁₋₆ alkyl;
R¹¹, R¹², R¹⁴, R¹⁷ and R¹⁹ are C₁₋₈ alkyl (optionally substituted by halo, hydroxy, C₁₋₆ alkoxy, C₁₋₆ haloalkoxy, C₃₋₆ cycloalkyl (optionally substituted by halo), C₅₋₆ cycloalkenyl, S(C₁₋₄ alkyl), S(O)(C₁₋₄ alkyl), S(O)₂(C₁₋₄ alkyl), heteroaryl, aryl, heteroaryloxy or aryloxy), aryl, heteroaryl, C₃₋₇ cycloalkyl (optionally substituted by halo, C₁₋₄ alkyl or C₁₋₄ haloalkyl), C₄₋₇ cycloalkyl fused to a phenyl ring, C₅₋₇ cycloalkenyl, or heterocyclyl; or R¹¹, R¹², R¹⁴ and R¹⁷ can also be hydrogen;
or R¹⁰ and R¹¹, and/or R¹⁶ and R¹⁷ may join to form a 4-, 5- or 6-membered ring which optionally includes a nitrogen, oxygen or sulphur atom, said ring being optionally substituted by C₁₋₆ alkyl, C₁₋₆ haloalkyl, S(O)ₗ(C₁₋₆ alkyl) or C(O)(C₁₋₆ alkyl);
R² is C₁₋₆ alkyl, phenyl, heteroaryl or C₃₋₇ cycloalkyl;
when X is NR⁵, Y is absent or is CH₂;
when X is CH₂, Y is absent, CH₂, NR⁶, O, S, S(O) or S(O)₂;
Z is a 5- or 6-membered heterocyclyl ring;
R³, R⁵ and R⁶ are, independently, hydrogen or C₁₋₆ alkyl;
R⁴ is hydrogen, C₁₋₄ alkyl, C₃₋₄ alkenyl, C₃₋₄ alkynyl or C₃₋₆ cycloalkyl; aryl, phenyl and heteroaryl moieties are independently optionally substituted by: halo, cyano, nitro, hydroxy, OC(O)NR²⁰R²¹, NR²²R²³, NR²⁴C(O)R²⁵, NR²⁶C(O)NR²⁷R²⁸, S(O)₂NR²⁹R³⁰, NR³¹S(O)₂R³², C(O)NR³³R³⁴, CO₂R³⁶, NR³⁷CO₂R³⁸, S(O)_{q}R³⁹, OS(O)₂R⁴⁹, C₁₋₆ alkyl (optionally mono-substituted by S(O)₂R⁵⁰ or C(O)NR⁵¹R⁵²), C₂₋₆ alkenyl, C₂₋₆ alkynyl, C₃₋₁₀ cycloalkyl, C₁₋₆ haloalkyl, C₁₋₆ alkoxy(C₁₋₆)alkyl, C₁₋₆ alkoxy (optionally mono-substituted by CO₂R⁵³, C(O)NR⁵⁴R⁵⁵, cyano, heteroaryl or C(O)NHS(O)₂R⁵⁶), NHC(O)NHR⁵⁷, C₁₋₆ haloalkoxy, phenyl, phenyl(C₁₋₄)alkyl, phenoxy, phenylthio, phenylS(O), phenylS(O)₂, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃;
unless otherwise stated heterocyclyl moieties are independently optionally substituted by: C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)} or heteroaryl {which itself optionally substituted by halo, C 4 alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, heteroaryl {optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, S(O)₂NR⁴⁰R⁴¹, C(O)R⁴², C(O)₂(C₁₋₆ alkyl) (such as tert-butoxycarbonyl), C(O)₂(phenyl(C₁₋₂ alkyl)) (such as benzyloxycarbonyl), C(O)NHR⁴³, S(O)₂R⁴⁴, NHS(O)₂NHR⁴⁵, NHC(O)R⁴⁶, NHC(O)NHR⁴⁷ or NHS(O)₂R⁴⁸, provided none of these last four substituents is linked to a ring nitrogen;
k, l, p and q are, independently, 0, 1 or 2;
R²⁰, R²², R²⁴, R²⁶, R²⁷, R²⁹, R³¹, R³³, R³⁷, R⁴⁰, R⁵¹ and R⁵⁴ are, independently, hydrogen or C₁₋₆ alkyl;
R²¹, R²³, R²⁵, R²⁸, R³⁰, R³², R³⁴, R³⁶, R³⁸, R³⁹, R⁴¹, R⁴², R⁴³, R⁴⁴, R⁴⁵, R⁴⁶, R⁴⁷, R⁴⁸, R⁴⁹, R⁵⁰, R⁵², R⁵³, R⁵⁵, R⁵⁶ and R⁵⁷ are, independently, C₁₋₆ alkyl (optionally substituted by halo, hydroxy, C₁₋₆ alkoxy, C₁₋₆ haloalkoxy, C₃₋₆ cycloalkyl, C₅₋₆ cycloalkenyl, S(C₁₋₄ alkyl), S(O)(C₁₋₄ alkyl), S(O)₂(C₁₋₄ alkyl), heteroaryl, phenyl, heteroaryloxy or phenyloxy), C₃₋₇ cycloalkyl, phenyl or heteroaryl; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
R²¹, R²³, R²⁵, R²⁸, R³⁰, R³⁴, R³⁵, R³⁶, R⁴¹, R⁴², R⁴³, R⁴⁵, R⁴⁶, R⁴⁷, R², R⁵³, and R⁵⁷ may additionally be hydrogen;
alternatively, R²⁰ and R²¹, and/or R²² and R²³, and/or R²⁷ and R²⁸, and/or R²⁹ and R³⁰, and/or R³³ and R³⁴, and/or R⁵¹ and R⁵² and/or R⁵⁴ and R⁵⁵, and/or R⁴⁰ and R⁴¹ may join to form a 5- or 6-membered ring which is optionally substituted with halo, C₁₋₄ alkyl or phenyl (wherein the phenyl ring is optionally substituted by halo, cyano, nitro, hydroxy, C₁₋₄ alkyl, C₁₋₄ alkoxy, S(O)ₘC₁₋₄ alkyl, S(O)₂NH₂, S(O)₂NH(C₁₋₄ alkyl), S(O)₂N(C₁₋₄ alkyl)₂, NHS(O)₂(C₁₋₄ alkyl), NH₂, NH(C₁₋₄ alkyl), N(C₁₋₄ alkyl)₂, NHC(O)NH₂, C(O)NH₂, C(O)NH(C₁₋₄ alkyl), NHC(O)(C₁₋₄ alkyl), CO₂H, CO₂(C₁₋₄ alkyl), C(O)(C₁₋₄ alkyl), CF₃, CHF₂, CH₂F, CH₂CF₃ or OCF₃);
m is 0, 1 or 2;
or a pharmaceutically acceptable salt thereof.

2. A compound of the formula (I) according to claim 1 wherein R¹ is heterocyclyl.

3. A compound of the formula (I) according to claim 1 or claim 2 wherein R¹ is piperidinyl or piperazinyl, either of which is N-substituted by phenyl, S(O)₂R³⁹ (wherein R³⁹ is C₁₋₄ alkyl, phenyl or CF₃) or S(O)₂NR²⁹R³⁰ (wherein R²⁹ and R³⁰ are, independently, C₁₋₄ alkyl).

4. A compound of the formula (I) according to claims 1 to 3 wherein R² is phenyl or heteroaryl, either of which is optionally substituted by halo, C₁₋₄ alkyl, C₁₋₄ alkoxy, S(O)ₙ(C₁₋₄ alkyl), nitro, cyano or CF₃; wherein n is 0, 1 or 2.

5. A compound of the formula (I) according to claims 1 to 4 wherein R⁴ is hydrogen, methyl, ethyl, n-propyl, allyl or cyclopropyl

6. A compound of the formula (I) according to claims 1 to 5 wherein Z is piperidinyl or piperazinyl, optionally substituted (such as on a ring nitrogen) by C(O)(C₁₋₆ alkyl), C(O)(C₁₋₆ alkoxy) or S(O)₂(C₁₋₄ alkyl).

7. A processes for preparation of a compound of the formula (I) as claimed in claim 1 which comprises:—
(a) reacting a compound of formula (II): 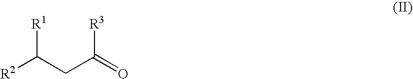
with a compound of formula (III): 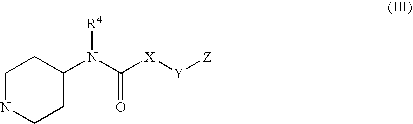
under reductive amination conditions, with a suitable organic acid and a suitable reducing agent; or
(b) reacting a compound of formula (IV): 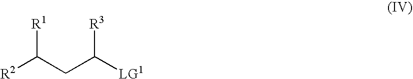
wherein the leaving group LG¹ is tosylate, mesylate, triflate or halogen; with a compound of formula (III); or
(c) reacting a compound of the formula (V): 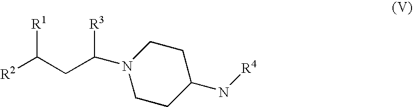
with:
when X is CH₂, a compound of formula (VI): 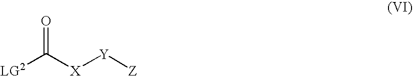
wherein LG² is halogen, an active ester or OH activated with a carbodiimide coupling agent, or the activated product of the reaction of an acid with carbonyldiimidazole; the reaction being carried out in an inert solvent in the presence of a base;
OR
when X is NH, a compound of formula (VII): 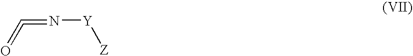
the reaction being carried out in an inert solvent in the presence of a base;
OR
when X is NR⁵, a compound of formula (VIII): 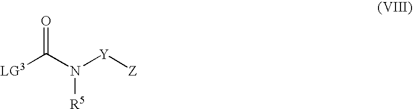
wherein LG³ is halogen or an active ester; the reaction being carried out in an inert solvent in the presence of a base;
wherein R¹, R², R³, R⁴ and R⁵ are as defined in claim 1.

8. A pharmaceutical composition which comprises a compound as claimed in claims 1 to 6, or a pharmaceutically acceptable salt thereof, and a pharmaceutically acceptable adjuvant, diluent or carrier.

9. A compound as claimed in claims 1 to 6, or a pharmaceutically acceptable salt thereof, for use as a medicament.

10. A compound as claimed in claims 1 to 6, or a pharmaceutically acceptable salt thereof, in the manufacture of a medicament for use in therapy.

11. A method of treating a CCR5 mediated disease state comprising administering to a patient in need of such treatment an effective amount of a compound as claimed in claims 1 to 6, or a pharmaceutically acceptable salt thereof.
